(19) United States
(12) Patent Application Publication
(10) Pub. No.: US 2010/0249098 A1
(43) Pub. Date: Sep. 30, 2010

(54) OXYPIPERIDINE DERIVATIVES AND METHODS OF USE THEREOF

(75) Inventors: Jianhua Chao, San Diego, CA (US); Robert G. Aslanian, Rockaway, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/680,282

(22) PCT Filed: Sep. 25, 2008
(86) PCT No.: PCT/US08/11111

§ 371 Date: Mar. 26, 2010

Related U.S. Application Data
(60) Provisional application No. 60/976,003, filed on Sep. 28, 2007.

(51) Int. Cl. A61K 31/4545; A61K 31/55; A61K 31/506; A61K 31/4725; A61K 31/4709; C07D 401/14; C07D 401/12; A61P 25/00; C07D 417/14; A61P 37/08; A61P 9/00; A61P 1/00; A61P 3/04; A61P 3/00

(52) U.S. Cl. 514/210.21; 514/210.2; 514/217.04; 514/275; 514/310; 514/314; 514/316; 514/318; 540/597; 544/332; 546/171; 546/187; 546/188

(57) ABSTRACT

The present invention relates to novel Oxypiperidine Derivatives, pharmaceutical compositions comprising the Oxypiperidine Derivatives and the use of the Oxypiperidine Derivatives for treating or preventing treating allergy, an allergy-induced airway response, congestion, hypotension, a cardiovascular disease, a gastrointestinal disorder, obesity, a sleep disorder, pain, diabetes, a diabetic complication, impaired glucose tolerance, impaired fasting glucose or a central nervous system (CNS) disorder.

### FIELD OF THE INVENTION

The present invention relates to novel Oxypiperidine Derivatives, pharmaceutical compositions comprising the Oxypiperidine Derivatives and the use of the Oxypiperidine Derivatives for treating or preventing treating allergy, an allergy-induced airway response, congestion, hypotension, a cardiovascular disease, a gastrointestinal disorder, obesity, a sleep disorder, pain, diabetes, a diabetic complication, impaired glucose tolerance, impaired fasting glucose or a central nervous system (CNS) disorder.

### BACKGROUND OF THE INVENTION

The histamine receptors, H₁, H₂ and H₃ are well-identified forms. The H₁ receptors are those that mediate the response antagonized by conventional antihistamines. H₁ receptors are present, for example, in the ileum, the skin, and the bronchial smooth muscle of humans and other mammals. Through H₂ receptor-mediated responses, histamine stimulates gastric acid secretion in mammals and the chronotropic effect in isolated mammalian atria.

H₃ receptor sites are found on sympathetic nerves, where they modulate sympathetic neurotransmission and attenuate a variety of end organ responses under control of the sympathetic nervous system. Specifically, H₃ receptor activation by histamine attenuates norepinephrine outflow to resistance and capacitance vessels, causing vasodilation.

Imidazole H₃ receptor antagonists are well known in the art. More recently, non-imidazole H₃ receptor antagonists have been disclosed in PCT US01/32151, filed Oct. 15, 2001, and U.S. Provisional Application 60/275,417, filed Mar. 13, 2001.

U.S. Pat. No. 5,869,479 discloses compositions for the treatment of the symptoms of allergic rhinitis using a combination of at least one histamine H₁ receptor antagonist and at least one histamine H₃ receptor antagonist.

Diabetes refers to a disease process derived from multiple causative factors and is characterized by elevated levels of plasma glucose, or hyperglycemia in the fasting state or after administration of glucose during an oral glucose tolerance test. Persistent or uncontrolled hyperglycemia is associated with increased and premature morbidity and mortality. Abnormal glucose homeostasis is associated with alterations of the lipid, lipoprotein and apolipoprotein metabolism and other metabolic and hemodynamic disease. As such, the diabetic patient is especially increased risk of macrovascular and microvascular complications, including coronary heart disease, stroke, peripheral vascular disease, hypertension, nephropathy, neuropathy, and retinopathy. Accordingly, therapeutic control of glucose homeostasis, lipid metabolism and hypertension are critically important in the clinical management and treatment of diabetes mellitus.

There are two generally recognized forms of diabetes. In type 1 diabetes, or insulin-dependent diabetes mellitus (IDDM), patients produce little or no insulin, the hormone which regulates glucose utilization. In type 2 diabetes, or noninsulin dependent diabetes mellitus (NIDDM), patients often have plasma insulin levels that are the same or even elevated compared to nondiabetic subjects; however, these patients have developed a resistance to the insulin stimulating effect on glucose and lipid metabolism in the main insulin-sensitive tissue (muscle, liver and adipose tissue), and the plasma insulin levels, while elevated, are insufficient to overcome the pronounced insulin resistance.

Insulin resistance is not associated with a diminished number of insulin receptors but rather to a post-insulin receptor binding defect that is not well understood. This resistance to insulin responsiveness results in insufficient insulin activation of glucose uptake, oxidation and storage in muscle, and inadequate insulin repression of lipolysis in adipose tissue and of glucose production and secretion in the liver.

The available treatments for type-2 diabetes, which have not changed substantially in many years, have recognized limitations. While physical exercise and reductions in dietary intake of calories will dramatically improve the diabetic condition, compliance with this treatment is very poor because of well-entrenched sedentary lifestyles and excess food consumption, especially of foods containing high amounts of saturated fat. Increasing the plasma level of insulin by administration of sulfonylureas (e.g. tolbutamide and glipizide) or meglitinide, which stimulate the pancreatic [beta]-cells to secrete more insulin, and/or by injection of insulin when sulfonylureas or meglitinide become ineffective, can result in insulin concentrations high enough to stimulate the very insulin-resistant tissues. However, dangerously low levels of plasma glucose can result from administration of insulin or insulin secretagogues (sulfonylureas or meglitinide), and an increased level of insulin resistance due to the even higher plasma insulin levels can occur. The biguanides are a class of agents that can increase insulin sensitivity and bring about some degree of correction of hyperglycemia. However, the biguanides can induce lactic acidosis and nausea/diarrhea.

The glitazones (i.e. 5-benzylthiazolidine-2,4-diones) are a separate class of compounds with potential for the treatment of type 2 diabetes. These agents increase insulin sensitivity in muscle, liver and adipose tissue in several animal models of type 2 diabetes, resulting in partial or complete correction of the elevated plasma levels of glucose without occurrence of hypoglycemia. The glitazones that are currently marketed are agonists of the peroxisome proliferator activated receptor (PPAR), primarily the PPAR-gamma subtype. PPAR-gamma agonism is generally believed to be responsible for the improved insulin sensitization that is observed with the glitazones. Newer PPAR agonists that are being tested for treatment of Type II diabetes are agonists of the alpha, gamma or delta subtype, or a combination of these, and in many cases are chemically different from the glitazones (i.e., they are not thiazolidinediones). Serious side effects (e.g. liver toxicity) have been noted in some patients treated with glitazone drugs, such as troglitazone.

Additional methods of treating the disease are currently under investigation. New biochemical approaches include treatment with alpha-glucosidase inhibitors (e.g. acarbose) and protein tyrosine phosphatase-1B (PTP-1B) inhibitors.

Compounds that are inhibitors of the dipeptidyl peptidase-IV enzyme are also under investigation as drugs that may be useful in the treatment of diabetes, and particularly type 2 diabetes.

Despite a widening body of knowledge in connection with discovery and use of histamine receptor modulators, there remains a need in the art for small-molecule histamine antagonists having increased safety profiles and/or improved efficacy. This invention addresses that need.

### SUMMARY OF THE INVENTION

The present invention provides compounds having the formula:

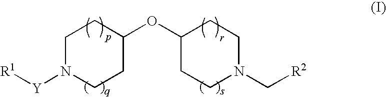

and pharmaceutically acceptable salts, solvates, esters and prodrugs thereof, wherein:

Y is a bond, -alkylene-, —C(O)—, —OC(O)— or —NHC(O)—;

R¹ is aryl, cycloalkyl, cycloalkenyl, heterocycloalkyl, heterocycloalkenyl or heteroaryl, wherein an aryl, cycloalkyl, cycloalkenyl, heterocycloalkyl, heterocycloalkenyl or heteroaryl group can be optionally substituted with up to 3 substituents, which can be the same or different, and are selected from alkyl, —O-alkyl, halo, haloalkyl, —O-haloalkyl, —CN, —C(O)OR³, —N(R⁴)₂, —C(O)N(R⁴)₂, —C(O)R⁵, —NHC(O)R⁵, —NHS(O)₂R³ or —S(O)₂N(R⁴)₂, and wherein R¹ is cycloalkyl, the cycloalkyl group can be optionally fused to an aryl or heteroaryl ring;

R² is aryl, heterocycloalkyl, heterocycloalkenyl or heteroaryl, wherein an aryl, heterocycloalkyl, heterocycloalkenyl or heteroaryl group can be optionally substituted with up to 3 substituents, which can be the same or different, and are selected from alkyl, cycloalkyl, heterocycloalkyl, heteroaryl, —O-alkyl, —O-aryl, halo, haloalkyl, —O-haloalkyl, —CN, —OC(O)R⁵, —C(O)OR³, —N(R⁴)₂, —C(O)N(R⁴)₂, —C(O)R⁵, —NHC(O)R⁵, —NHS(O)₂R³ or —S(O)₂N(R⁴)₂;

each occurrence of R³ is independently H, alkyl, aryl, cycloalkyl, heterocycloalkyl or heteroaryl;

each occurrence of R⁴ is independently H, alkyl, aryl, cycloalkyl, heterocycloalkyl or heteroaryl;

each occurrence of R⁵ is independently H, alkyl, aryl, cycloalkyl, cycloalkenyl, heterocycloalkyl, heterocycloalkenyl, heteroaryl, —O-alkyl, —NH-alkyl, —O-aryl or —NH-aryl;

p is an integer ranging from 0 to 2;

q is an integer ranging from 0 to 2;

r is an integer ranging from 0 to 2; and

s is an integer ranging from 0 to 2.

The compounds of formula (I) (the “Oxypiperidine Derivatives”), and pharmaceutically acceptable salts, solvates, esters and prodrugs thereof, are useful for treating or preventing allergy, an allergy-induced airway response, congestion, hypotension, a cardiovascular disease, a gastrointestinal disorder, obesity, a sleep disorder, pain, diabetes, a diabetic complication, impaired glucose tolerance, impaired fasting glucose or a central nervous system (CNS) disorder (each being a “Condition”) in a patient.

This invention also provides pharmaceutical compositions comprising an effective amount of at least one Oxypiperidine Derivative and a pharmaceutically acceptable carrier.

This invention further provides methods for treating or preventing a Condition in a patient, comprising administering to the patient an effective amount of one or more Oxypiperidine Derivatives or a pharmaceutically acceptable salt, solvate, ester or prodrug thereof.

### DETAILED DESCRIPTION OF THE INVENTION

As used above, and throughout this disclosure, the following terms, unless otherwise indicated, shall be understood to have the following meanings:

A “patient” is a human or non-human mammal. In one embodiment, a patient is a human. In another embodiment, a patient is a non-human mammal, including, but not limited to, a monkey, dog, baboon, rhesus, mouse, rat, horse, cat or rabbit. In another embodiment, a patient is a companion animal, including but not limited to a dog, cat, rabbit, horse or ferret. In one embodiment, a patient is a dog. In another embodiment, a patient is a cat.

The term “obesity” as used herein, refers to a patient being overweight and having a body mass index (BMI) of 25 or greater. In one embodiment, an obese patient has a BMI of 25 or greater. In another embodiment, an obese patient has a BMI from 25 to 30. In another embodiment, an obese patient has a BMI greater than 30. In still another embodiment, an obese patient has a BMI greater than 40.

The term “impaired glucose tolerance” as used herein, is defined as a two-hour glucose level of 140 to 199 mg per dL (7.8 to 11.0 mmol) as measured using the 75-g oral glucose tolerance test. A patient is said to be under the condition of impaired glucose tolerance when he/she has an intermediately raised glucose level after 2 hours, wherein the level is less than would qualify for type 2 diabetes mellitus.

The term “impaired fasting glucose” as used herein, is defined as a fasting plasma glucose level of 100 to 125 mg/dL; normal fasting glucose values are below 100 mg per dL.

The term “upper airway” as used herein, refers to the upper respiratory system—i.e., the nose, throat, and associated structures.

The term “effective amount” as used herein, refers to an amount of Oxypiperidine Derivative and/or an additional therapeutic agent, or a composition thereof that is effective in producing the desired therapeutic, ameliorative, inhibitory or preventative effect when administered to a patient suffering from a Condition. In the combination therapies of the present invention, an effective amount can refer to each individual agent or to the combination as a whole, wherein the amounts of all agents administered are together effective, but wherein the component agent of the combination may not be present individually in an effective amount.

In one embodiment, the compounds of this invention can be ligands for the histamine H₃ receptor. In another embodiment, the compounds of this invention can also be described as antagonists of the H₃ receptor, or as “H₃ antagonists.”

The term “alkyl,” as used herein, refers to an aliphatic hydrocarbon group which may be straight or branched and which contains from about 1 to about 20 carbon atoms. In one embodiment, an alkyl group contains from about 1 to about 12 carbon atoms. In another embodiment, an alkyl group contains from about 1 to about 6 carbon atoms. Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is unsubstituted. In another embodiment, an alkyl group is linear. In another embodiment, an alkyl group is branched.

The term “alkenyl,” as used herein, refers to an aliphatic hydrocarbon group containing at least one carbon-carbon double bond and which may be straight or branched and contains from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkenyl group is unsubstituted.

The term “alkynyl,” as used herein, refers to an aliphatic hydrocarbon group containing at least one carbon-carbon triple bond and which may be straight or branched and contains from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-aryl, —O—C(O)-cycloalkyl, —C(O)OH and —C(O)O-alkyl. In one embodiment, an alkynyl group is unsubstituted.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and —CH₂CH(CH₃)CH₂—. In one embodiment, an alkylene group has from 1 to about 6 carbon atoms. In another embodiment, an alkylene group is branched. In another embodiment, an alkylene group is linear.

The term “aryl,” as used herein, refers to an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. Non-limiting examples of aryl groups include phenyl and naphthyl. In one embodiment, an aryl group is unsubstituted. In another embodiment, an aryl group is phenyl.

The term “cycloalkyl,” as used herein, refers to a non-aromatic mono- or multicyclic ring system comprising from about 3 to about 10 ring carbon, atoms. In one embodiment, a cycloalkyl contains from about 5 to about 10 ring carbon atoms. In another embodiment, a cycloalkyl contains from about 5 to about 7 ring atoms. The term “cycloalkyl” also encompasses a cycloalkyl group, as defined above, that is fused to an aryl (e.g., benzene) or heteroaryl ring. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. In one embodiment, a cycloalkyl group is unsubstituted.

The term “cycloalkenyl,” as used herein, refers to a non-aromatic mono- or multicyclic ring system comprising from about 3 to about 10 ring carbon atoms and containing at least one endocyclic double bond. In one embodiment, a cycloalkenyl contains from about 5 to about 10 ring carbon atoms. In another embodiment, a cycloalkenyl contains 5 or 6 ring atoms. Non-limiting examples of monocyclic cycloalkenyls include cyclopentenyl, cyclohexenyl, cyclohepta-1,3-dienyl, and the like. A cycloalkenyl group can be optionally substituted with one or more “ring system substituents” which may be the same or different, and are as defined herein below. In one embodiment, a cycloalkenyl group is unsubstituted. In another embodiment, a cycloalkenyl group is a 5-membered cycloalkenyl.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. A heteroaryl group can be optionally substituted by one or more “ring system substituents” which may be the same or different, and are as defined herein below. A heteroaryl group is joined via a ring carbon atom, and any nitrogen atom of a heteroaryl can be optionally oxidized to the corresponding N-oxide. The term “heteroaryl” also encompasses a heteroaryl group, as defined above, that is fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like. In one embodiment, a heteroaryl group is unsubstituted. In another embodiment, a heteroaryl group is a 5-membered heteroaryl. In another embodiment, a heteroaryl group is a 6-membered heteroaryl.

The term “heterocycloalkyl,” as used herein, refers to a non-aromatic saturated monocyclic or multicyclic ring system comprising 3 to about 10 ring atoms, wherein from 1 to 4 of the ring atoms are independently O, S or N and the remainder of the ring atoms are carbon atoms. In one embodiment, a heterocycloalkyl group has from about 5 to about 10 ring atoms. In another embodiment, a heterocycloalkyl group has 5 or 6 ring atoms. There are no adjacent oxygen and/or sulfur atoms present in the ring system. Any —NH group in a heterocycloalkyl ring may exist protected such as, for example, as an —N(BOC), —N(Cbz), —N(Tos) group and the like; such protected heterocycloalkyl groups are considered part of this invention. The term “heterocycloalkyl” also encompasses a heterocycloalkyl group, as defined above, that is fused to an aryl (e.g., benzene) or heteroaryl ring. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. Non-limiting examples of monocyclic heterocycloalkyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. A ring carbon atom of a heterocycloalkyl group may be functionalized as a carbonyl group. An illustrative example of such a heterocycloalkyl group is pyrrolidonyl:

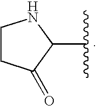

In one embodiment, a heterocycloalkyl group is unsubstituted. In another embodiment, a heterocycloalkyl group is a 5-membered heterocycloalkyl. In another embodiment, a heterocycloalkyl group is a 6-membered heterocycloalkyl.

The term “heterocycloalkenyl,” as used herein, refers to a heterocycloalkyl group, as defined above, wherein the heterocycloalkyl group contains from 3 to 10 ring atoms, and at least one endocyclic carbon-carbon or carbon-nitrogen double bond. In one embodiment, a heterocycloalkenyl group has from 5 to 10 ring atoms. In another embodiment, a heterocycloalkenyl group is monocyclic and has 5 or 6 ring atoms. A heterocycloalkenyl group can optionally substituted by one or more ring system substituents, wherein “ring system substituent” is as defined above. The nitrogen or sulfur atom of the heterocycloalkenyl can be optionally oxidized to the corresponding N-oxide, S-oxide or S,S-dioxide. Non-limiting examples of heterocycloalkenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluoro-substituted dihydrofuranyl, 7-oxabicyclo[2.2.1]heptenyl, dihydrothiophenyl, dihydrothiopyranyl, and the like. A ring carbon atom of a heterocycloalkenyl group may be functionalized as a carbonyl group. An illustrative example of such a heterocycloalkenyl group is:

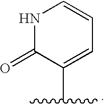

In one embodiment, a heterocycloalkenyl group is unsubstituted. In another embodiment, a heterocycloalkenyl group is a 5-membered heterocycloalkenyl.

The term “5-membered heterocycloalkenyl,” as used herein, refers to a heterocycloalkenyl group, as defined above, which has 5 ring atoms.

It should also be noted that tautomeric forms such as, for example, the moieties:

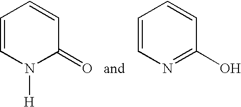

are considered equivalent in certain embodiments of this invention.

The term “ring system substituent,” as used herein, refers to a substituent group attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, -alkyl-aryl, -aryl-alkyl, -alkylene-heteroaryl, -alkenylene-heteroaryl, -alkynylene-heteroaryl, hydroxy, hydroxyalkyl, haloalkyl, —O-alkyl, —O-haloalkyl, -alkylene-O-alkyl, -D-aryl, aralkoxy, acyl, aroyl, halo, nitro, cyano, carboxy, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and -alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

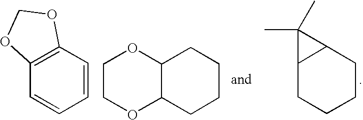

“Halo” means —F, —Cl, —Br or —I. In one embodiment, halo refers to —F, —Cl or —Br.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂Cl and —CCl₃.

The term “hydroxyalkyl,” as used herein, refers to an alkyl group as defined above, wherein one or more of the alkyl group's hydrogen atoms has been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃.

The term “alkoxy” as used herein, refers to an —O-alkyl group, wherein an alkyl group is as defined above. Non-limiting examples of alkoxy groups include methoxy, ethoxy, n-propoxy, isopropoxy, n-butoxy and t-butoxy. An alkoxy group is bonded via its oxygen atom.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “purified”, “in purified form” or “in isolated and purified form” for a compound refers to the physical state of the compound after being isolated from a synthetic process (e.g. from a reaction mixture), or natural source or combination thereof. Thus, the term “purified”, “in purified form” or “in isolated and purified form” for a compound refers to the physical state of the compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any variable (e.g., aryl, heterocycle, R², etc.) occurs more than one time in any constituent or in Formula (I), its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield an Oxypiperidine Derivative or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if an Oxypiperidine Derivative or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if an Oxypiperidine Derivative contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If an Oxypiperidine Derivative incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl, carboxy(C₁-C₆)alkyl, amino(C₁-C₄)alkyl or mono-N- or di-N,N-(C₁-C₆)alkylaminoalkyl, —C(Y⁴)Y⁵ wherein Y⁴ is H or methyl and Y⁵ is mono-N- or di-N,N-(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The Oxypiperidine Derivatives can form salts which are also within the scope of this invention. Reference to an Oxypiperidine Derivative herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when an Oxypiperidine Derivative contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the compounds of the Formula (I) may be formed, for example, by reacting an Oxypiperidine Derivative with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (for example, methyl, ethyl, n-propyl, isopropyl, t-butyl, sec-butyl or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Stereochemically pure compounds may also be prepared by using chiral starting materials or by employing salt resolution techniques. Also, some of the Oxypiperidine Derivatives may be atropisomers (e.g., substituted biaryls) and are considered as part of this invention. Enantiomers can also be separated by use of chiral HPLC column.

It is also possible that the Oxypiperidine Derivatives may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if an Oxypiperidine Derivative incorporates a double bond or a fused ring, both the cis- and trans-forms, as well as mixtures, are embraced within the scope of the invention. Also, for example, all keto-enol and imine-enamine forms of the compounds are included in the invention).

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled Oxypiperidine Derivatives (e.g., those labeled with ³H and ¹⁴C) are useful in compound and/or substrate tissue distribution assays. In one embodiment, tritiated (i.e., ³H) and carbon-14 (i.e., ¹⁴C) isotopes are employed for their ease of preparation and detectability. In another embodiment, substitution with heavier isotopes such as deuterium (i.e., ²H) may afford certain therapeutic advantages resulting from greater metabolic stability (e.g., increased in vivo half-life or reduced dosage requirements). Isotopically labelled

### Oxypiperidine Derivatives

Synthetic chemical procedures analogous to those disclosed herein for making the Oxypiperidine Derivatives, by substituting an appropriate isotopically labelled starting material or reagent for a non-isotopically labelled starting material or reagent.

Polymorphic forms of the Oxypiperidine Derivatives, and of the salts, solvates, hydrates, esters and prodrugs of the Oxypiperidine Derivatives, are intended to be included in the present invention.

The following abbreviations are used below and have the following meanings: Ac is acetyl, Boc or BOC is —C(O)O-(t-butyl), t-butyl is tertiary butyl, DIAD is diisopropylazodicarboxylate, DMF is N,N-dimethylformamide, DMSO is dimethylsulfoxide, EtOAc is ethyl acetate, EtOH is ethanol, LCMS is liquid chromatography mass spectrometry, MeOH is methanol, NaOEt is sodium ethoxide, NaOtBu is sodium t-butoxide, NMR is nuclear magnetic resonance, Ph is phenyl, Ph₃P is triphenylphosphine, TFA is tetrafluoroacetic acid, THF is tetrahydrofuran, TLC is thin-layer chromatography and TsOH is p-toluenesulfonic acid.

### The Oxypiperidine Derivatives of Formula (I)

In one embodiment, the invention provides Oxypiperidine Derivatives of formula (I):

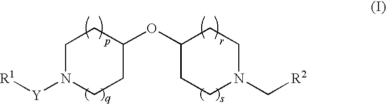

and pharmaceutically acceptable salts and solvates thereof, wherein R¹, R², p, q, r and s are defined above for the compounds of formula (I).

In one embodiment, Y is a bond.

In another embodiment, Y is alkylene.

In another embodiment, Y is —C(O)—.

In still another embodiment, —OC(O)—.

In yet another embodiment, Y is —NHC(O)—.

In a further embodiment, Y is a bond, —CH₂, or —C(O)—.

In another embodiment, Y is —CH₂—.

In one embodiment, R¹ is aryl.

In another embodiment, R¹ is cycloalkyl.

In another embodiment, R¹ is cycloalkenyl.

In still another embodiment, R¹ is heterocycloalkyl.

In yet another embodiment, R¹ is heterocycloalkenyl.

In another embodiment, R¹ is heteroaryl.

In another embodiment, R¹ is cycloalkyl, aryl or heteroaryl.

In still another embodiment, R¹ is a 6-membered heteroaryl.

In yet another embodiment, R¹ is pyridyl.

In another embodiment, R¹ is pyrimidinyl.

In another embodiment, R¹ is phenyl.

In a further embodiment, R¹ is cycloalkyl which is fused to a heteroaryl group.

In another embodiment, R¹ is cycloalkyl which is fused to a pyridyl group.

In another embodiment, R¹ is cycloalkyl which is fused to a benzene group.

In one embodiment, R¹ is:

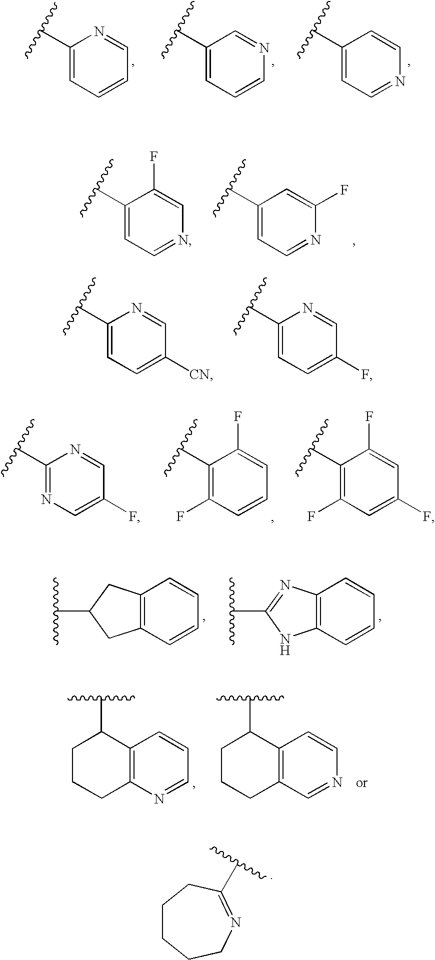

In one embodiment, R² is aryl.

In another embodiment, R² is heterocycloalkyl.

In another embodiment, R² is heterocycloalkenyl.

In still another embodiment, R² is heteroaryl.

In another embodiment, R² is heteroaryl or heterocycloalkyl.

In another embodiment, R² is 5-membered heteroaryl.

In yet another embodiment, R² is 6-membered heteroaryl.

In one embodiment, R² is 5- or 6-membered heteroaryl.

In another embodiment, R² is 4-membered heterocycloalkyl.

In another embodiment, R² is 5-membered heterocycloalkyl.

In yet another embodiment, R² is 6-membered heterocycloalkyl.

In one embodiment, R² is pyridyl.

In another embodiment, R² is pyridin-4-yl.

In another embodiment, R² is pyridyl substituted with —N(R⁴)₂.

In another embodiment, R² is pyridyl substituted with —NH₂.

In still another embodiment, R² is thiazolyl.

In yet another embodiment, R² is thiazolyl substituted with —N(R⁴)₂.

In another embodiment, R² is thiazolyl substituted with —NH₂.

In a further embodiment, R² is 4-membered heterocycloalkyl.

In one embodiment, R² is:

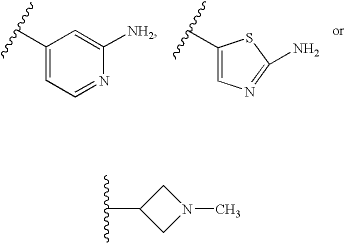

In one embodiment, the sum of p and q is 1.

In another embodiment, the sum of p and q is 2.

In another embodiment, the sum of p and q is 3.

In still another embodiment, p is 1.

In another embodiment, q is 1.

In still another embodiment, p is 2.

In another embodiment, q is 2.

In yet another embodiment, p and q are each 1.

In one embodiment, the sum of r and s is 1.

In another embodiment, the sum of r and s is 2.

In another embodiment, the sum of r and s is 3.

In still another embodiment, r is 1.

In another embodiment, s is 1.

In still another embodiment, r is 2.

In another embodiment, s is 2.

In yet another embodiment, r and s are each 1.

In a further embodiment, p, q, r and s are each 1.

In one embodiment, R¹ is cycloalkyl, aryl or heteroaryl and R² is heteroaryl or heterocycloalkyl.

In one embodiment, R¹ is cycloalkyl, aryl or heteroaryl and R² is heteroaryl or heterocycloalkyl.

In another embodiment, R¹ is heteroaryl and R² is heteroaryl.

In another embodiment, Y is -alkylene-, R¹ is heteroaryl and R² is heteroaryl.

In another embodiment, Y is —C(O)—, R¹ is heteroaryl and R² is heteroaryl.

In still another embodiment, Y is —NHC(O)—, R¹ is heteroaryl and R² is heteroaryl.

In another embodiment, R¹ is cycloalkyl and R² is heteroaryl.

In another embodiment, Y is -alkylene-, R¹ is aryl and R² is heteroaryl.

In still another embodiment, Y is —CH₂—, R¹ is heteroaryl and R² is heteroaryl.

In another embodiment, Y is —CH₂—, R¹ is pyridyl and R² is heteroaryl.

In yet another embodiment, Y is —CH₂—, R¹ is aryl and R² is heteroaryl.

In still another embodiment, Y is —CH₂—, R¹ is phenyl and R² is heteroaryl.

In another embodiment, R¹ is cycloalkenyl and R² is heteroaryl.

In one embodiment, R¹ is heteroaryl and R² is 5- or 6-membered heteroaryl.

In another embodiment, Y is alkylene, R¹ is heteroaryl and R² is 5- or 6-membered heteroaryl.

In another embodiment, Y is —C(O)—, R¹ is heteroaryl and R² is 5- or 6-membered heteroaryl.

In still another embodiment, Y is —NHC(O)—, R¹ is heteroaryl and R² is 5- or 6-membered heteroaryl.

In another embodiment, R¹ is cycloalkyl and R² is 5- or 6-membered heteroaryl.

In another embodiment, Y is alkylene, R¹ is aryl and R² is 5- or 6-membered heteroaryl.

In still another embodiment, Y is —CH₂—, R¹ is heteroaryl and R² is 5- or 6-membered heteroaryl.

In another embodiment, R¹ is cycloalkenyl and R² is 5- or 6-membered heteroaryl.

In one embodiment, R¹ is pyridyl and R² is 5- or 6-membered heteroaryl.

In another embodiment, Y is —CH₂—, R¹ is pyridyl and R² is 5- or 6-membered heteroaryl.

In another embodiment, Y is —C(O)—, R¹ is pyridyl and R² is 5- or 6-membered heteroaryl.

In still another embodiment, Y is —NHC(O)—, R¹ is pyridyl and R² is 5- or 6-membered heteroaryl.

In another embodiment, R¹ is cyclohexyl and R² is 5- or 6-membered heteroaryl.

In one embodiment, R¹ is heteroaryl and R² is pyridyl or thiazolyl.

In another embodiment, Y is alkylene, R¹ is heteroaryl and R² is 5- or 6-membered heteroaryl.

In another embodiment, Y is —C(O)—, R¹ is heteroaryl and R² is pyridyl or thiazolyl.

In still another embodiment, Y is —NHC(O)—, R¹ is heteroaryl and R² is pyridyl or thiazolyl.

In another embodiment, R¹ is cycloalkyl and R² is pyridyl or thiazolyl.

In another embodiment, Y is alkylene, R¹ is aryl and R² is pyridyl or thiazolyl.

In still another embodiment, Y is —CH₂—, R¹ is aryl and R² is pyridyl or thiazolyl.

In another embodiment, R¹ is cycloalkenyl and R² is pyridyl or thiazolyl.

In one embodiment, R¹ is pyridyl and R² is pyridyl or thiazolyl.

In another embodiment, Y is —CH₂—, R¹ is pyridyl and R² is pyridyl or thiazolyl.

In another embodiment, Y is —C(O)—, R¹ is pyridyl and R² is pyridyl or thiazolyl.

In still another embodiment, Y is —NHC(O)—, R¹ is pyridyl and R² is pyridyl or thiazolyl.

In another embodiment, R¹ is cyclohexyl and R² is pyridyl or thiazolyl.

In a further embodiment, Y is —CH₂—, R¹ is phenyl and R² is pyridyl or thiazolyl.

In another embodiment, R¹ is -cycloalkyl fused to a heteroaryl and R² is pyridyl or thiazolyl.

In another embodiment, Y is —C(O)—, R¹ is phenyl and R² is pyridyl or thiazolyl.

In still another embodiment, Y is —NHC(O)—, R¹ is pyridyl and R² is pyridyl or thiazolyl.

In one embodiment, R² is heteroaryl or heterocycloalkyl and R¹ is:

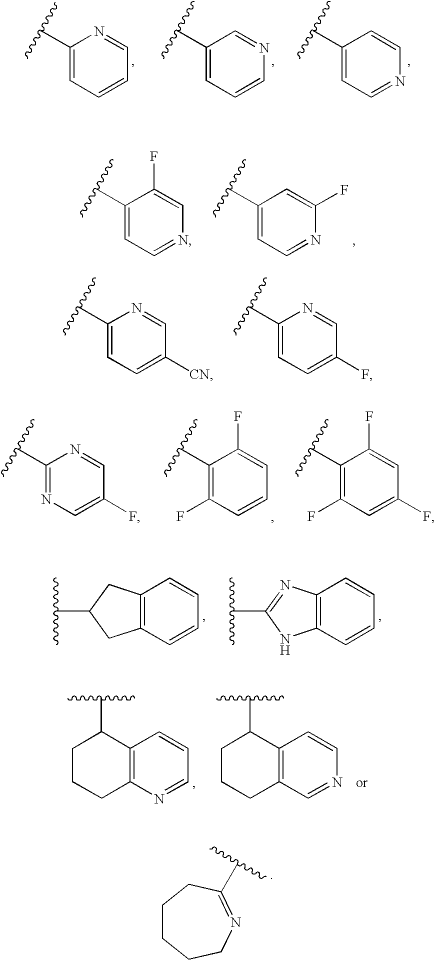

In another embodiment, R¹ is:

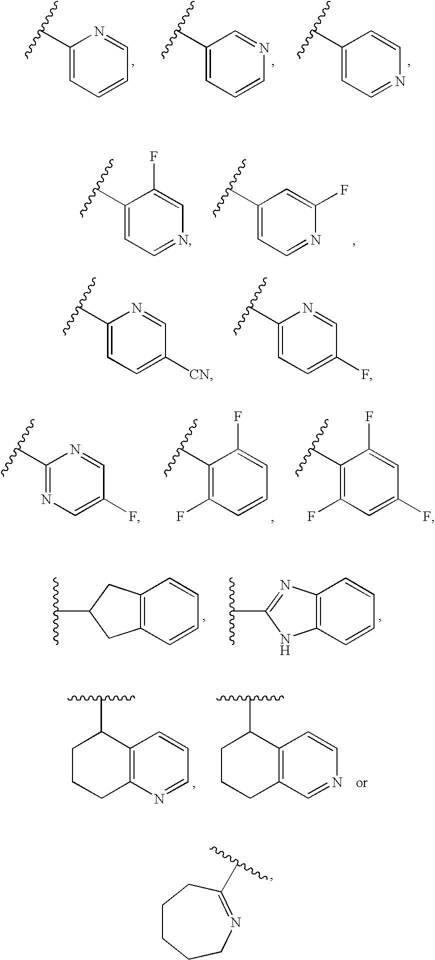

and R² is:

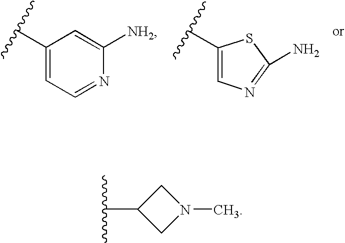

In one embodiment, Y is a bond, —CH₂, or —C(O)—, and R¹ is cycloalkyl, aryl or heteroaryl.

In another embodiment, Y is a bond, —CH₂, or —C(O)—; R¹ is cycloalkyl, aryl or heteroaryl; and R² is heteroaryl or heterocycloalkyl.

In another embodiment, Y is a bond, —CH₂, or —C(O)—; R¹ is:

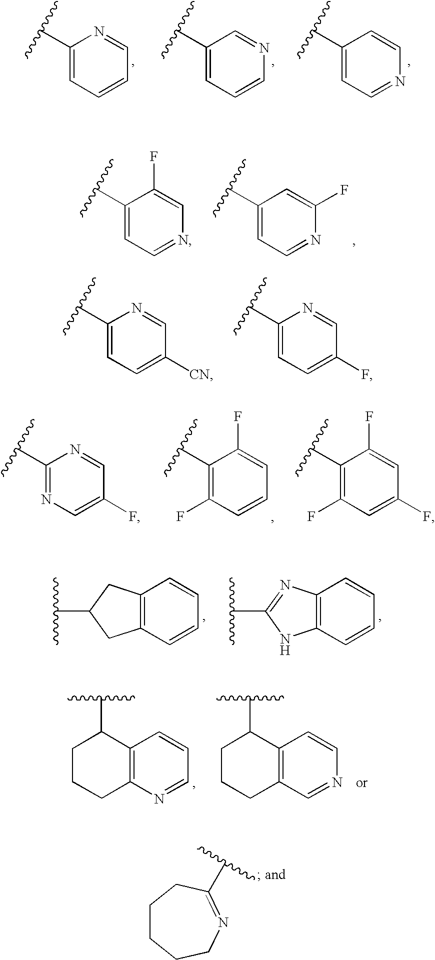

R² is heteroaryl or heterocycloalkyl.

In another embodiment, Y is a bond, —CH₂, or —C(O)—; R¹ is:

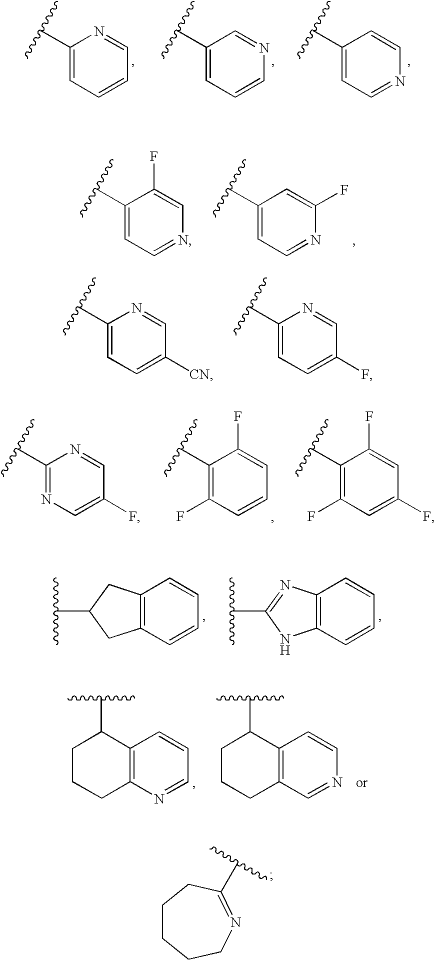

and R² is:

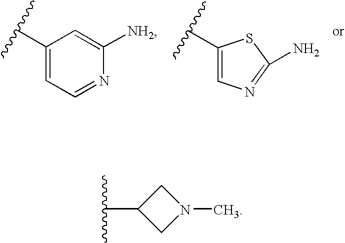

In still another embodiment, p is 1; q is 1; Y is a bond, —CH₂, or —C(O)—; R¹ is cycloalkyl, aryl or heteroaryl; and R² is heteroaryl or heterocycloalkyl.

In yet another embodiment, p is 1; q is 1; Y is a bond, —CH₂, or —C(O)—; R¹ is:

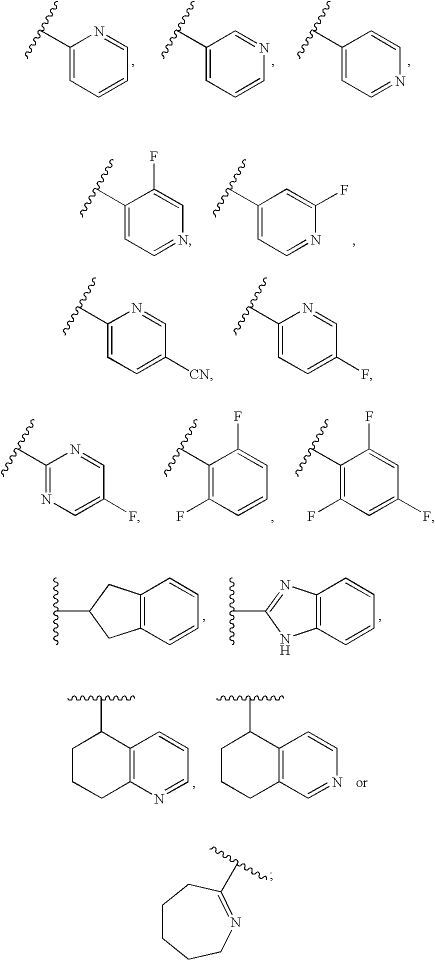

and R² is:

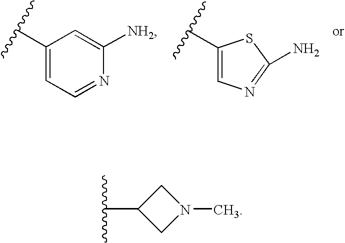

In one embodiment, a compound of formula (I) is in purified form.

In one embodiment, the compounds of formula (I) have the formula (II):

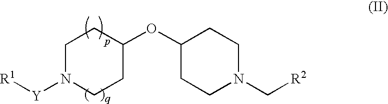

wherein:

Y is a bond, -alkylene-, —C(O)— or —NHC(O)—;

R¹ is aryl, cycloalkyl, heterocycloalkyl or heteroaryl, wherein an aryl, cycloalkyl, heterocycloalkyl or heteroaryl group can be optionally substituted with up to 3 substituents, which can be the same or different, and are selected from alkyl, halo, haloalkyl, —CN and —N(R⁴)₂;

R² is heterocycloalkyl or heteroaryl, either of which can be optionally substituted with up to 3 substituents, which can be the same or different, and are selected from alkyl, halo, haloalkyl, —CN and —N(R⁴)₂;

each occurrence of R⁴ is independently H or alkyl;

p is an integer ranging from 0 to 2; and

q is an integer ranging from 0 to 2.

In one embodiment, Y is a bond.

In another embodiment, Y is alkylene.

In another embodiment, Y is —C(O)—.

In yet another embodiment, Y —NHC(O)—.

In a further embodiment, Y is a bond, —CH₂, or —C(O)—.

In another embodiment, Y is —CH₂—.

In one embodiment, R¹ is aryl.

In another embodiment, R¹ is cycloalkyl.

In still another embodiment, R¹ is heterocycloalkyl.

In another embodiment, R¹ is heteroaryl.

In still another embodiment, R¹ is a 6-membered heteroaryl.

In yet another embodiment, R¹ is pyridyl.

In another embodiment, R¹ is pyrimidinyl.

In another embodiment, R¹ is phenyl.

In a further embodiment, R¹ is cycloalkyl which is fused to a heteroaryl group.

In another embodiment, R¹ is cycloalkyl which is fused to a pyridyl group.

In another embodiment, R¹ is cycloalkyl which is fused to a benzene group.

In one embodiment, R¹ is:

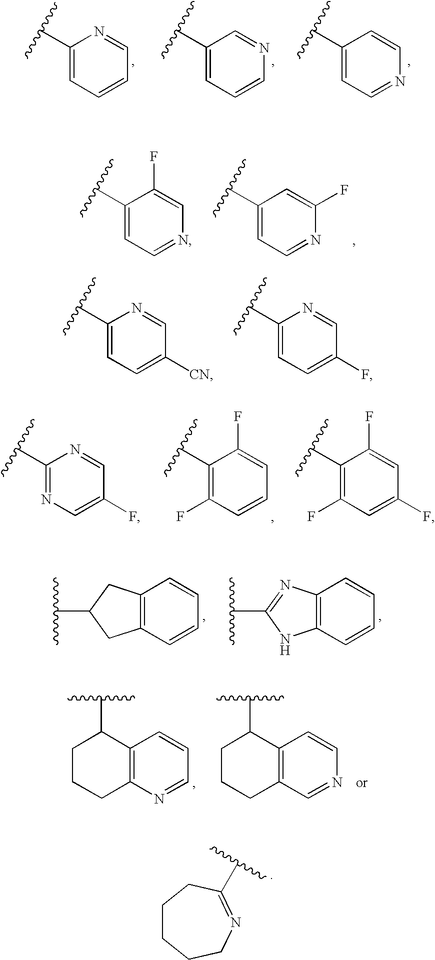

In one embodiment, R² is heterocycloalkyl.

In still another embodiment, R² is heteroaryl.

In another embodiment, R² is 5-membered heteroaryl.

In yet another embodiment, R² is 6-membered heteroaryl.

In another embodiment, R² is 4-membered heterocycloalkyl.

In another embodiment, R² is 5-membered heterocycloalkyl.

In yet another embodiment, R² is 6-membered heterocycloalkyl.

In one embodiment, R² is pyridyl.

In another embodiment, R² is pyridin-4-yl.

In another embodiment, R² is pyridyl substituted with —N(R⁴)₂.

In another embodiment, R² is pyridyl substituted with —NH₂.

In still another embodiment, R² is thiazolyl.

In yet another embodiment, R² is thiazolyl substituted with —N(R⁴)₂.

In another embodiment, R² is thiazolyl substituted with —NH₂.

In a further embodiment, R² is 4-membered heterocycloalkyl.

In one embodiment, R² is:

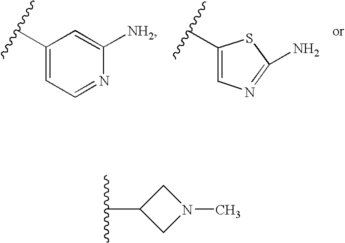

In one embodiment, the sum of p and q is 1.

In another embodiment, the sum of p and q is 2.

In another embodiment, the sum of p and q is 3.

In still another embodiment, p is 1.

In another embodiment, q is 1.

In still another embodiment, p is 2.

In another embodiment, q is 2.

In yet another embodiment, p and q are each 1.

In one embodiment, R¹ is heteroaryl and R² is heteroaryl.

In another embodiment, Y is -alkylene-, R¹ is heteroaryl and R² is heteroaryl.

In another embodiment, Y is —C(O)—, R¹ is heteroaryl and R² is heteroaryl.

In still another embodiment, Y is —NHC(O)—, R¹ is heteroaryl and R² is heteroaryl.

In another embodiment, R¹ is cycloalkyl and R² is heteroaryl.

In another embodiment, Y is -alkylene-, R¹ is aryl and R² is heteroaryl.

In still another embodiment, Y is —CH₂—, R¹ is heteroaryl and R² is heteroaryl.

In another embodiment, Y is —CH₂—, R¹ is pyridyl and R² is heteroaryl.

In yet another embodiment, Y is —CH₂—, R¹ is aryl and R² is heteroaryl.

In still another embodiment, Y is —CH₂—, R¹ is phenyl and R² is heteroaryl.

In another embodiment, R¹ is cycloalkenyl and R² is heteroaryl.

In one embodiment, R¹ is heteroaryl and R² is 5- or 6-membered heteroaryl.

In another embodiment, Y is alkylene, R¹ is heteroaryl and R² is 5- or 6-membered heteroaryl.

In another embodiment, Y is —C(O)—, R¹ is heteroaryl and R² is 5- or 6-membered heteroaryl.

In still another embodiment, Y is —NHC(O)—, R¹ is heteroaryl and R² is 5- or 6-membered heteroaryl.

In another embodiment, R¹ is cycloalkyl and R² is 5- or 6-membered heteroaryl.

In another embodiment, Y is alkylene, R¹ is aryl and R² is 5- or 6-membered heteroaryl.

In still another embodiment, Y is —CH₂—, R¹ is heteroaryl and R² is 5- or 6-membered heteroaryl.

In another embodiment, R¹ is cycloalkenyl and R² is 5- or 6-membered heteroaryl.

In one embodiment, R¹ is pyridyl and R² is 5- or 6-membered heteroaryl.

In another embodiment, Y is —CH₂—, R¹ is pyridyl and R² is 5- or 6-membered heteroaryl.

In another embodiment, Y is —C(O)—, R¹ is pyridyl and R² is 5- or 6-membered heteroaryl.

In still another embodiment, Y is —NHC(O)—, R¹ is pyridyl and R² is 5- or 6-membered heteroaryl.

In another embodiment, R¹ is cyclohexyl and R² is 5- or 6-membered heteroaryl.

In one embodiment, R¹ is heteroaryl and R² is pyridyl or thiazolyl.

In another embodiment, Y is alkylene, R¹ is heteroaryl and R² is 5- or 6-membered heteroaryl.

In another embodiment, Y is —C(O)—, R¹ is heteroaryl and R² is pyridyl or thiazolyl.

In still another embodiment, Y is —NHC(O)—, R¹ is heteroaryl and R² is pyridyl or thiazolyl.

In another embodiment, R¹ is cycloalkyl and R² is pyridyl or thiazolyl.

In another embodiment, Y is alkylene, R¹ is aryl and R² is pyridyl or thiazolyl.

In still another embodiment, Y is —CH₂—, R¹ is aryl and R² is pyridyl or thiazolyl.

In another embodiment, R¹ is cycloalkenyl and R² is pyridyl or thiazolyl.

In one embodiment, R¹ is pyridyl and R² is pyridyl or thiazolyl.

In another embodiment, Y is —CH₂—, R¹ is pyridyl and R² is pyridyl or thiazolyl.

In another embodiment, Y is —C(O)—, R¹ is pyridyl and R² is pyridyl or thiazolyl.

In still another embodiment, Y is —NHC(O)—, R¹ is pyridyl and R² is pyridyl or thiazolyl.

In another embodiment, R¹ is cyclohexyl and R² is pyridyl or thiazolyl.

In another embodiment, Y is —CH₂—, R¹ is phenyl and R² is pyridyl or thiazolyl.

In a further embodiment, R¹ is -cycloalkyl fused to a heteroaryl and R² is pyridyl or thiazolyl.

In another embodiment, Y is —C(O)—, R¹ is phenyl and R² is pyridyl or thiazolyl.

In still another embodiment, Y is —NHC(O)—, R¹ is pyridyl and R² is pyridyl or thiazolyl.

In one embodiment, R² is heteroaryl or heterocycloalkyl and R¹ is:

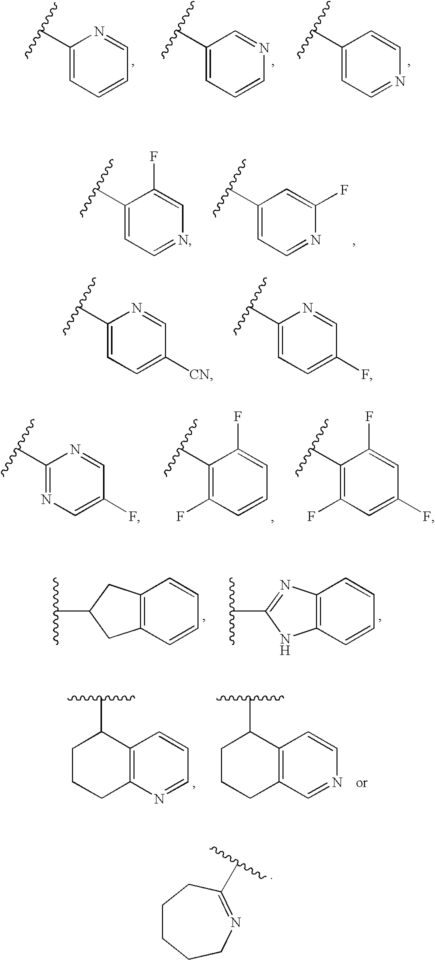

In another embodiment, R¹ is:

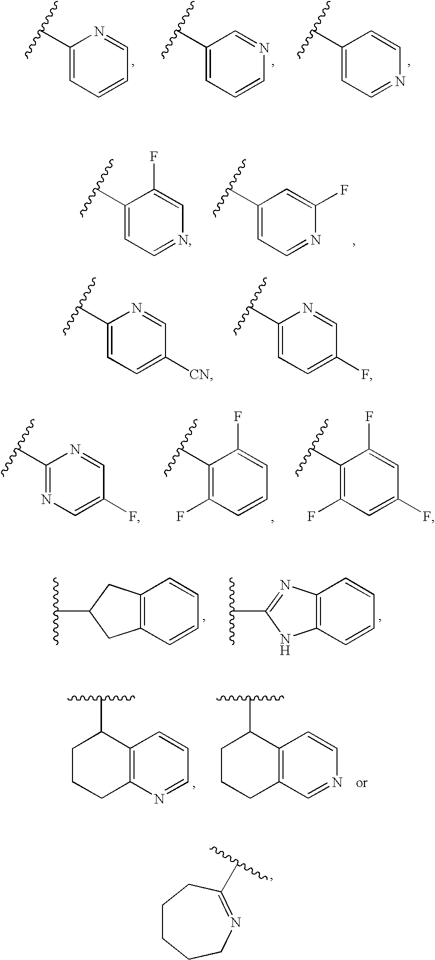

and R² is:

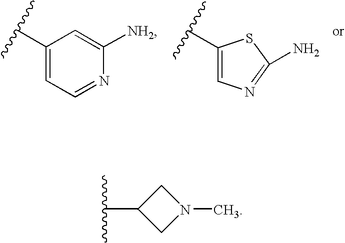

In one embodiment, Y is a bond, —CH₂, or —C(O)—, and R¹ is cycloalkyl, aryl or heteroaryl.

In another embodiment, Y is a bond, —CH₂, or —C(O)—; R¹ is cycloalkyl, aryl or heteroaryl; and R² is heteroaryl or heterocycloalkyl.

In another embodiment, Y is a bond, —CH₂, or —C(O)—; R¹ is:

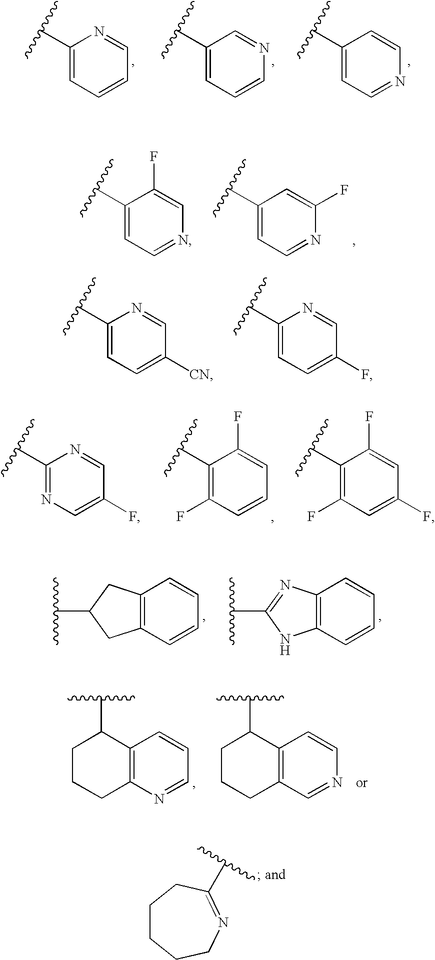

R² is heteroaryl or heterocycloalkyl.

In another embodiment, Y is a bond, —CH₂, or —C(O)—; R¹ is:

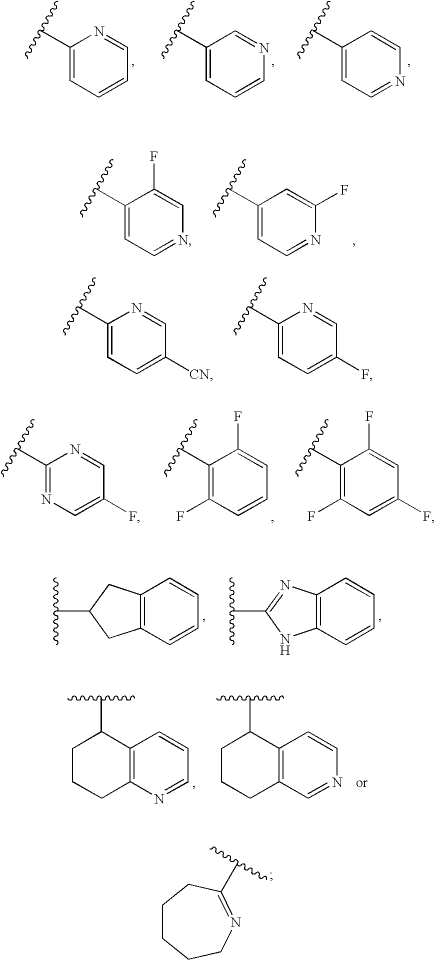

and R² is:

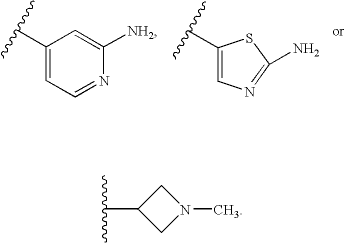

In still another embodiment, p is 1; q is 1; Y is a bond, —CH₂, or —C(O)—; R¹ is cycloalkyl, aryl or heteroaryl; and R² is heteroaryl or heterocycloalkyl.

In yet another embodiment, p is 1; q is 1; Y is a bond, —CH₂, or —C(O)—; R¹ is:

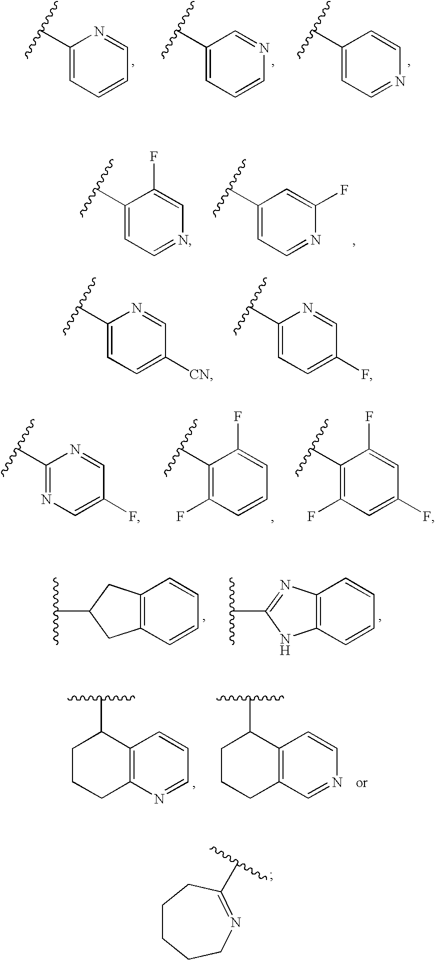

and R² is:

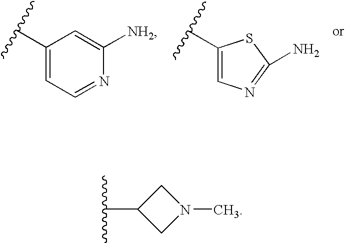

In one embodiment, a compound of formula (II) is in purified form.

Non-limiting illustrative examples of the Oxypiperidine Derivatives of formula (I) include the compounds in the following table:

| Compound | |
|---|---|
| No. | Structure |
| 1 | 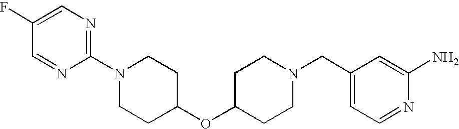 |
| 2 | 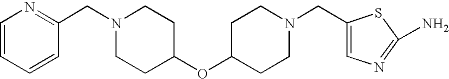 |
| 3 | 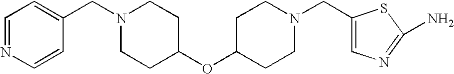 |
| 4 | 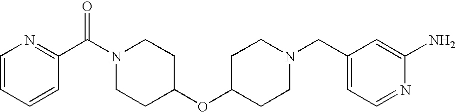 |
| 5 | 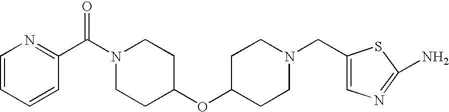 |
| 6 | 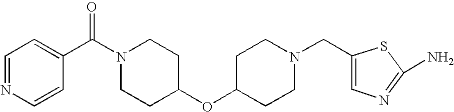 |
| 7 | 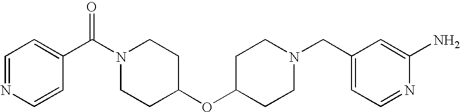 |
| 8 | 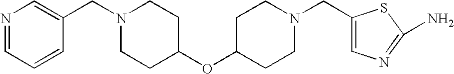 |
| 9 | 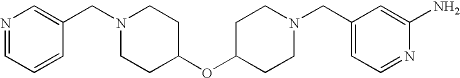 |
| 10 | 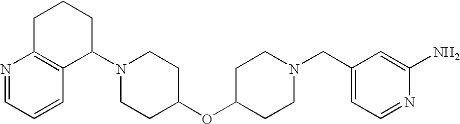 |
| 11 | 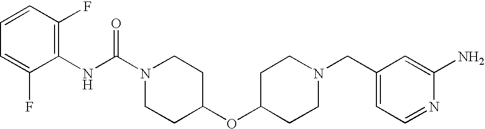 |
| 12 | 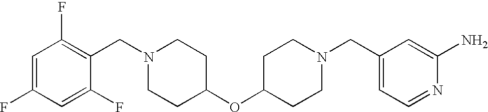 |
| 13 | 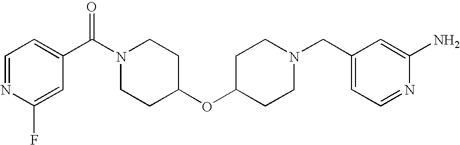 |
| 14 | 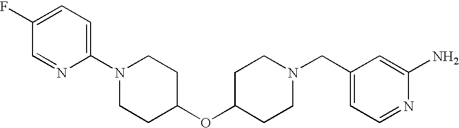 |
| 15 | 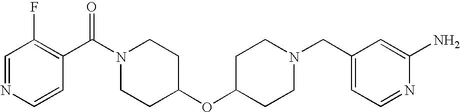 |
| 16 | 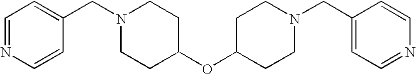 |
| 17 | 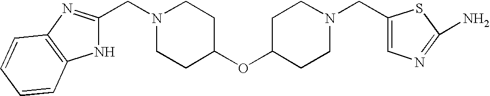 |
| 18 | 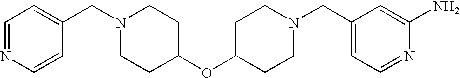 |
| 19 | 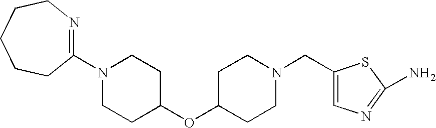 |
| 20 | 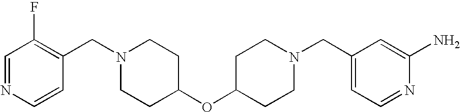 |
| 21 | 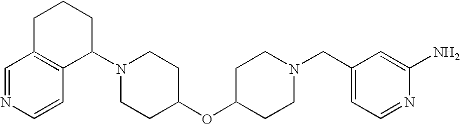 |
| 22 | 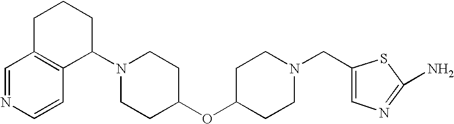 |
| 23 | 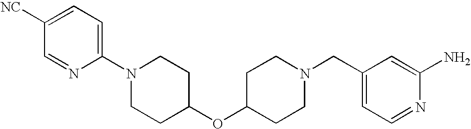 |
| 24 | 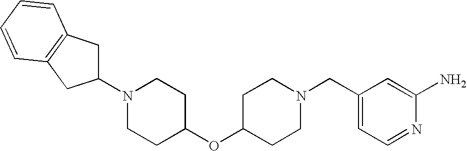 |
| 25 | 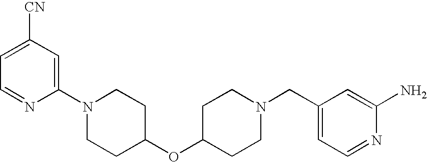 |
| 26 | 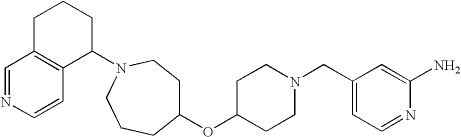 |
| 27 | 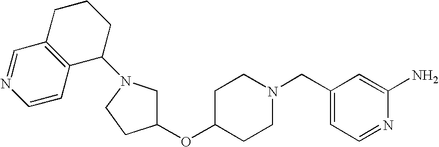 |
| 28 | 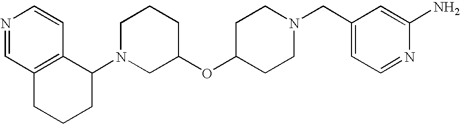 |
| 29 | 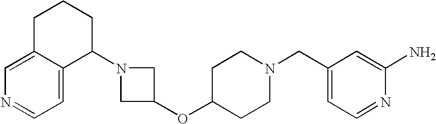 |

and pharmaceutically acceptable salts, solvates, esters and prodrugs thereof.

### Methods for Making the Oxypiperidine Derivatives

Methods useful for making the Oxypiperidine Derivatives are set forth in the Examples below and generalized in Schemes 1-7. Alternative synthetic pathways and analogous structures will be apparent to those skilled in the art of organic synthesis.

Scheme 1 shows how to make compounds of formula iv, which are useful intermediates for making the Oxypiperidine Derivatives.

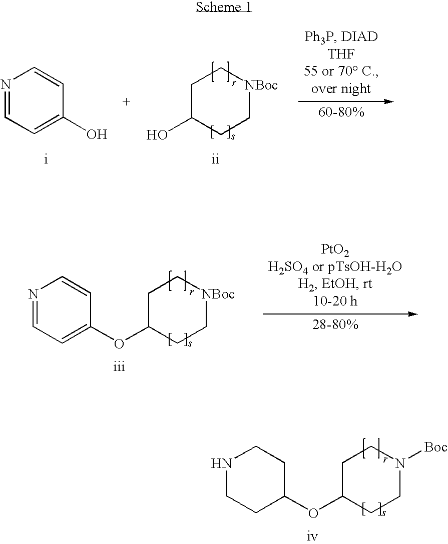

wherein r and s are defined above for the compounds of formula (I).

4-hydroxypyridine (i) can be coupled with a hydroxypiperidine compound of formula II via a Mitsunobu reaction to provide the oxypyridine compounds of formula iii. The pyridyl moiety of a compound of formula in can then be reduced using the conditions outlined in Scheme 1 to provide the corresponding oxypiperidine compounds of formula iv.

Scheme 2 illustrates methods useful for converting the intermediates of formula iv to the Oxypiperidine Derivatives, wherein Y is alkylene.

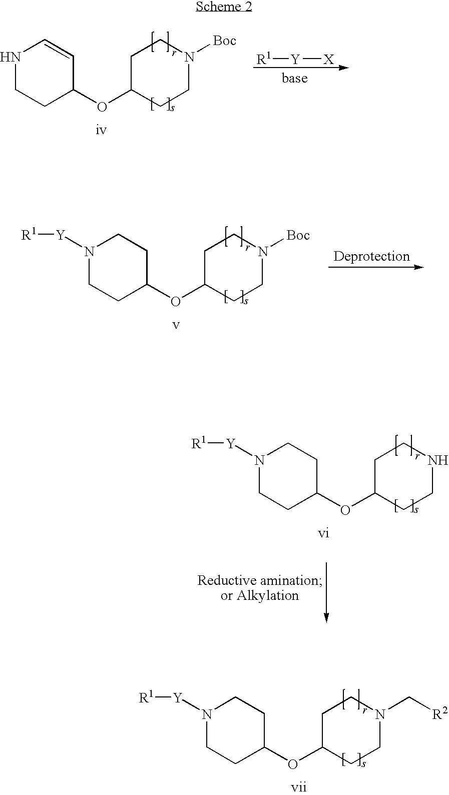

wherein R¹, R², r and s are defined above for the compounds of formula (I), Y is alkylene, and X is a good leaving group, such as —Cl, —Br, —I, —O-mesyl, —O-tosyl or —O-triflyl.

A compound of formula iv can be reacted with a compound of formula R¹—Y—X under mild basic conditions to provide the compounds of formula v. The Boc protecting group of a compound of formula v can than be removed using TFA, for example, to provide a compound of formula vi, which can then be: (1) reacted with a suitable aldehyde or ketone via a reductive amination process or (2) reacted with an alkylating agent via an alkylation process to provide the compound of formula vii, which correspond to the Oxypiperidine Derivatives of formula (I) wherein Y is an alkylene group.

Scheme 3 illustrates methods useful for converting the intermediates of formula iv to the Oxypiperidine Derivatives, wherein Y is —C(O)—.

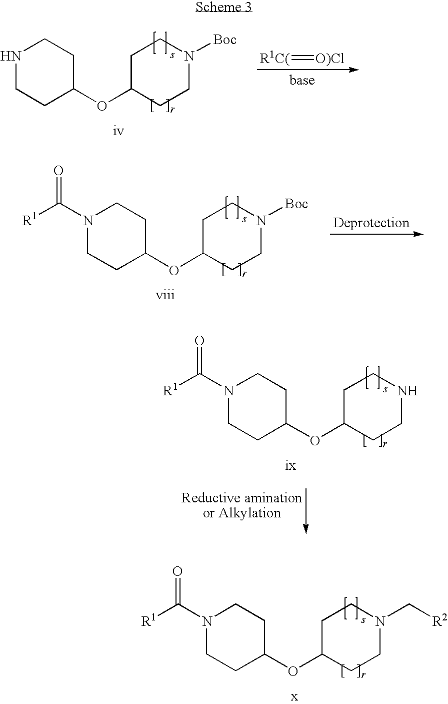

wherein R¹, R², r and s are defined above for the compounds of formula (I).

A compound of formula iv can be reacted with an acid chloride compound of formula R¹—C(O)Cl under mild basic conditions to provide the compounds of formula viii. The Boc protecting group of a compound of formula viii can then be removed using TFA, for example, to provide a compound of formula ix, which can then be: (1) reacted with a suitable aldehyde or ketone via a reductive amination process or (2) reacted with an alkylating agent via an alkylation process to provide the compounds of formula x, which correspond to the Oxypiperidine Derivatives of formula (I) wherein Y is —C(O)—.

Scheme 4 illustrates methods useful for converting the intermediates of formula iv to the Oxypiperidine Derivatives, wherein Y is —NHC(O)—.

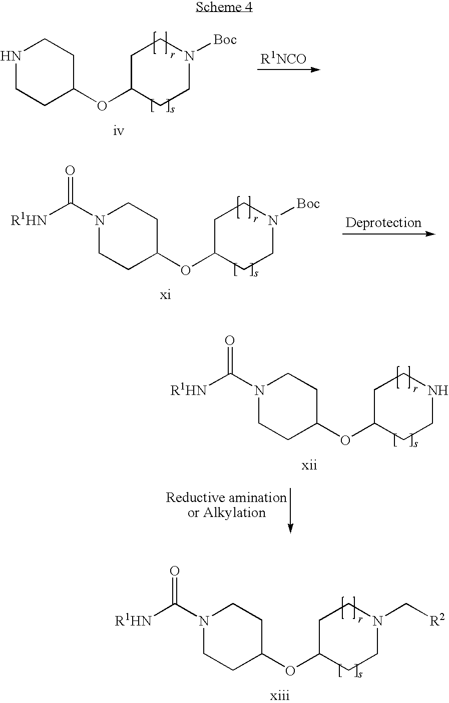

wherein R¹, R², r and s are defined above for the compounds of formula (I).

A compound of formula iv can be reacted with an isocyanate compound of formula R¹NCO to provide the compounds of formula xi. The Boc protecting group of a compound of formula xi can than be deprotected using TFA, for example, to provide a compound of formula xii, which can then be: (1) reacted with a suitable aldehyde or ketone via a reductive amination process or (2) reacted with an alkylating agent via an alkylation process to provide the compounds of formula xiii, which correspond to the Oxypiperidine Derivatives of formula (I) wherein Y is —NHC(O)—.

Scheme 5 illustrates methods useful for converting the intermediates of formula iv to the Oxypiperidine Derivatives, wherein Y is a bond and R¹ is aryl or heteroaryl.

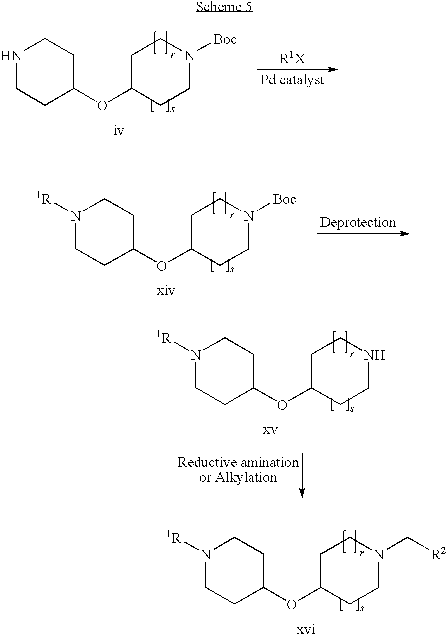

wherein R¹, R², r and s are defined above for the compounds of formula (I) and X is a good leaving group, such as —Cl, —Br, —I, —O-mesyl, —O-tosyl or —O-triflyl.

A compound of formula iv can undergo a palladium-catalyzed coupling with a compound of formula R¹—X to provide the compounds of formula xiv. Such coupling reactions are well-known in the art. The Boc protecting group of a compound of formula xiv can then be deprotected using TFA, for example, to provide a compound of formula xv, which can then be: (1) reacted with a suitable aldehyde or ketone via a reductive amination process or (2) reacted with an alkylating agent via an alkylation process to provide the compounds of formula xvi, which correspond to the Oxypiperidine Derivatives of formula (I) wherein Y is a bond and R¹ is aryl or heteroaryl.

Scheme 6 illustrates methods useful for converting the intermediates of formula iv to the Oxypiperidine Derivatives, wherein Y is a bond and R¹ is cycloalkyl, cycloalkylene or heterocycloalkyl.

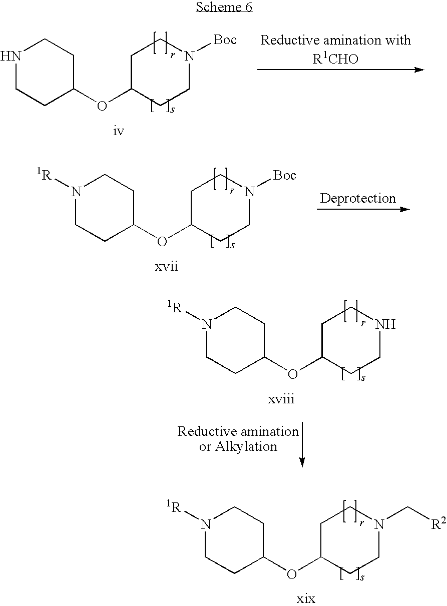

wherein R¹, R², r and s are defined above for the compounds of formula (I).

A compound of formula iv can be reacted with a compound of formula R¹CHO or via a reductive amination process to provide the compounds of formula xvii. Boc protecting group of a compound of formula xvii can than be removed, using TFA for example, to provide a compound of formula xviii, which can then be: (1) reacted with a suitable aldehyde or ketone via a reductive amination process or (2) reacted with an alkylating agent via an alkylation process to provide the compounds of formula xix, which correspond to the Oxypiperidine Derivatives of formula (I), wherein Y is a bond and R¹ is cycloalkyl, cycloalkylene or heterocycloalkyl.

Scheme 7 illustrates an alternative method useful making the Oxypiperidine Derivatives.

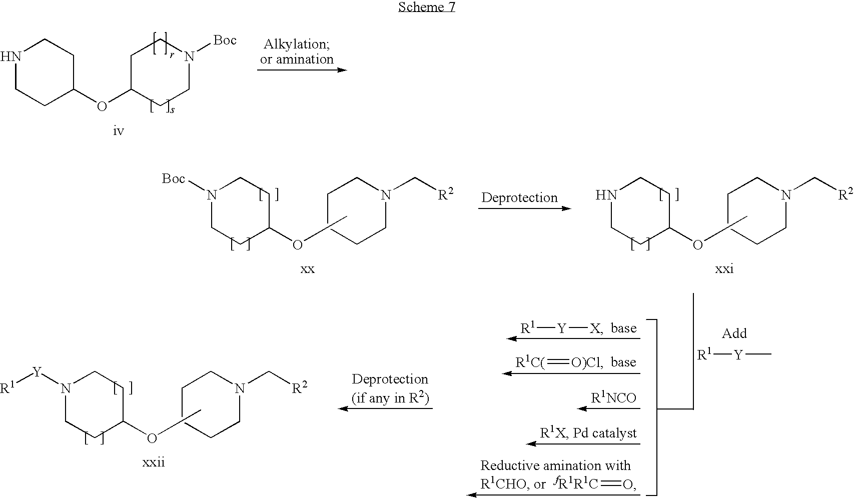

The Oxypiperidine Derivatives of Formula (I) may also be prepared from compounds of formula iv by first derivatizing a piperidine nitrogen atom of iv with a —CH₂—R² group using an alkylation process or reductive amination process to provide the compounds of formula xx. The Boc protecting group of the compounds of formula xx can then be removed using trifluoroacetic acid or iodotrimethylsilane, for example, to provide a compound of formula xxi. The free amine group of the compounds of formula xxi can then be derivatized as described above in any of Schemes 2-6 to provide the compounds of formula xxii, which correspond to the Oxypiperidine Derivatives of Formula (I).

The starting materials and reagents depicted in Schemes 1-7 are either available from commercial suppliers such as Sigma-Aldrich (St. Louis, Mo.) and Acros Organics Co. (Fair Lawn, N.J.), or can be prepared using methods well-known to those of skilled in the art of organic synthesis.

One skilled in the art will recognize that the synthesis of Oxypiperidine Derivatives may require the need for the protection of certain functional groups (i.e., derivatization for the purpose of chemical compatibility with a particular reaction condition). Suitable protecting groups for the various functional groups of the Oxypiperidine Derivatives and methods for their installation and removal may be found in Greene et al., Protective Groups in Organic Synthesis, Wiley-Interscience, New York, (1999).

### EXAMPLES

The following examples exemplify illustrative examples of compounds of the present invention and are not to be construed as limiting the scope of the disclosure. Alternative mechanistic pathways and analogous structures within the scope of the invention may be apparent to those skilled in the art.

### General Methods

The starting materials and reagents used in preparing compounds described are either available from commercial suppliers such as Aldrich Chemical Co. (Wisconsin, USA) and Acros Organics Co. (New Jersey, USA) or were prepared using methods well-known to those skilled in the art of organic synthesis. All commercially purchased solvents and reagents were used as received. LCMS analysis was performed using an Applied Biosystems API-100 mass spectrometer equipped with a Shimadzu SCL-10A LC column: Altech platinum C18, 3 um, 33 mm×7 mm ID; gradient flow: 0 minutes, 10% CH₃CN; 5 minutes, 95% CH₃CN; 7 minutes, 95% CH₃CN; 7.5 minutes, 10% CH₃CN; 9 minutes, stop. Flash column chromatography was performed using Selecto Scientific flash silica gel, 32-63 mesh. Analytical and preparative TLC was performed using Analtech Silica gel GF plates. Chiral HPLC was performed using a Varian PrepStar system equipped with a Chiralpak OD column (Chiral Technologies).

### Example 1

### Preparation of Compound 4

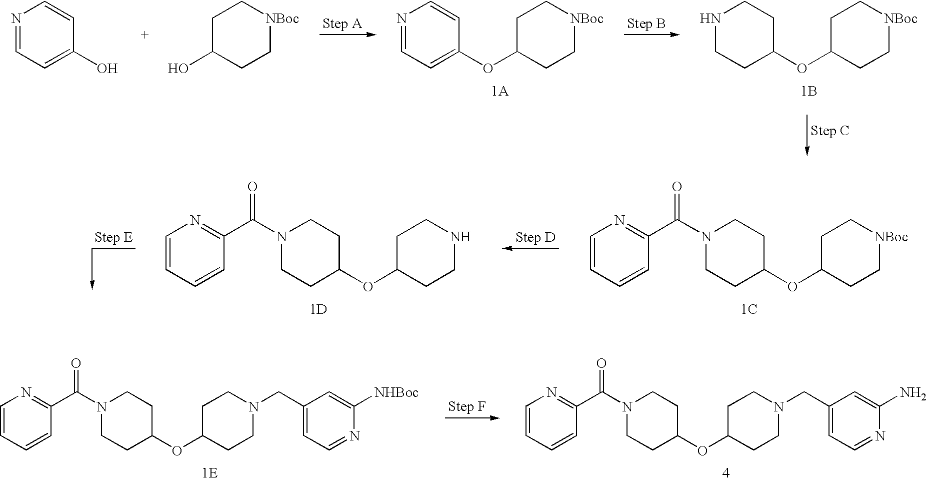

### Step A

To a stirred solution of 4-hydroxypyridine (2 g, 21.03 mmol) in 70 mL of anhydrous THF at room temperature was added 4-hydroxypiperidine (5.29 g, 26.28 mmol). Triphenyl phosphine (6.9 g, 26.31 mmol) was then added followed by dropwise addition of diisopropylazodicarboxylate (5.2 mL, 26.41 mmol). The reaction was heated to 55° C. and allowed to stir at this temperature for about 15 hours. The reaction mixture was then cooled to RT and concentrated in vacuo. The resulting oily residue was treated with a 1.0 M HCl aqueous solution (30 mL), and the acidic solution was washed with CH₂Cl₂ (30 mL×2). The combined CH₂Cl₂ washings were re-extracted with a 1.0 M HCl aqueous solution (10 mL) and H₂O (20 mL), then discarded. The aqueous fractions were combined, basified to pH ˜12 using a 1.0 M NaOH aqueous solution, and the basic solution was extracted with CH₂Cl₂ (50 mL×4). The combined organic extracts were washed with brine, dried over anhydrous Na₂SO₄, then concentrated in vacuo to provide a crude residue, which was purified using flash column chromatography, eluting with EtOAc-Hexanes-MeOH (5:1:0.1, v/v/v) to provide 3.92 g of pyridyl ether 1A (67%, MH⁺=279.2).

### Step B

Pyridyl ether 1A (1 g, 3.6 mmol) was dissolved in 20 mL of absolute ethanol. The solution was degassed under vacuum, and placed under nitrogen atmosphere. Platinum oxide (0.25 g, 0.25 weight equiv.) was added and the resulting mixture was degassed again, then placed under nitrogen atmosphere. Concentrated sulfuric acid (0.19 mL, 3.6 mmol) was added, the reaction was degassed a third time, and then put under H₂ atmosphere using a gas-filled balloon. The reaction was allowed to stir at room temperature for about 14 hours and was then poured into 50 mL of an ice cold 1.0 M NaOH aqueous solution. The resulting solution was rinsed with a small volume of CH₂Cl₂, and filtered through a Celite® pad. The organic solvent was removed in vacuo and the remaining aqueous solution was extracted with CH₂Cl₂ (50 mL×3). The combined organic extracts were washed with brine, dried over Na₂SO₄, and concentrated in vacuo to provide an oily residue, which was purified using flash column chromatography, eluting with CH₂Cl₂-MeOH (10:1, 5:1, and 1:1, v/v) to provide 0.61 g of bis-N-heterocylic alkyl ether 1B (60%, MH⁺=285.21).

### Step C

To a stirred solution of compound 1B (180 mg, 0.633 mmol) in 7 mL of CH₂Cl₂ was added triethylamine (0.31 mL, 2.22 mmol), followed by picolinoyl chloride hydrochloride (141 mg, 0.792 mmol). The reaction was allowed to stir at RT for about 60 hours, then was diluted with CH₂Cl₂ (60 mL), washed sequentially with saturated aqueous NaHCO₃ solution, then brine, dried over Na₂SO₄, and concentrated in vacuo. The oily residue obtained was purified using preparative TLC(CH₂Cl₂-7N NH₃ in MeOH=30:1, v/v) to provide 167 mg of the amide 1C (68%, MH⁺=390.2).

### Step D

Amide 1C (166 mg, 0.426 mmol) was dissolved in 3 mL of CH₂Cl₂ and to the resulting solution was added trifluoroacetic acid (0.5 mL). The reaction was allowed to stir for 2.5 hours at room temperature, then 1.0 M NaOH aqueous solution was added (15 mL). The resulting mixture was extracted with CH₂Cl₂ (25 mL×2) and the combined organic extracts were washed with brine, dried over Na₂SO₄, and concentrated in vacuo to provide 87 mg of the piperidyl piperidine intermediate 1D (71%, MH⁺=290.11).

### Step E

Piperidyl piperidine intermediate 1D (42 mg, 0.143 mmol) was dissolved in 2 mL of CH₂Cl₂. 2-Boc-amino-4-formal pyridine (42 mg, 0.189 mmol) was then added followed by sodium triacetoxyborohydride (40 mg, 0.189 mmol) and a catalytic amount of acetic acid was added. The reaction was allowed to stir at RT for about 15 hours, then H₂O was added, the aqueous mixture was extracted with CH₂Cl₂ (20 mL×3), and the combined organic extracts were washed with brine, dried over Na₂SO₄ and concentrated in vacuo to provide 85.6 mg of the product 1E (crude, MH⁺=496.21).

### Step F

Compound 1E (85.6 mg) was dissolved in a mixture of 2 mL CH₂Cl₂ and 0.5 mL trifluoroacetic acid. The mixture was stirred at room temperature for 16 hours, then 1.0 M NaOH aqueous solution was added. The aqueous mixture was extracted with CH₂Cl₂ (30 mL×3) and the combined organic extracts were washed with brine, dried over Na₂SO₄, and concentrated in vacuo to provide a crude oil, which was purified using preparative TLC (CH₂Cl₂-7N NH₃ in MeOH=30:1, v/v) to provide 37 mg of compound 4 (65%, MH⁺=396.2).

### Example 2

### Preparation of Intermediate Compound 2B

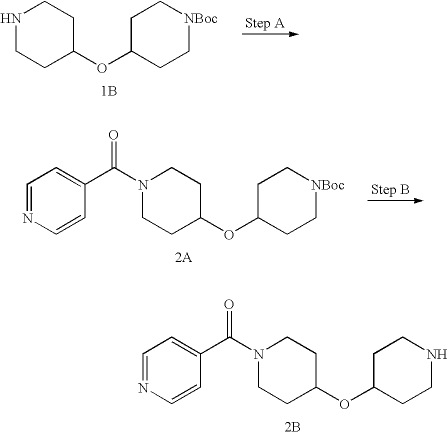

### Step A

Using the method described in Example 1 Step C, bis-N-heterocylic alkyl ether 1B (196 mg, 0.689 mmol) was converted to compound 2A (155 mg, 58%).

### Step B

Using the method described in Example 1 Step D, amide 2A (155 mg) was converted to compound 2B (96.4 mg, 86.5%).

### Examples 3 and 4

### Preparation of Intermediate Compounds 3A and 4A

Using the methods described in Example 2, except using the acid specified in the table below, compound 1B was converted to the intermediate compounds 3A and 4A.

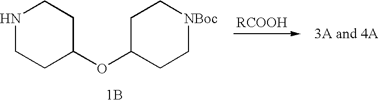

| Example | | | Yield (%)/ |
|---|---|---|---|
| No. | RCOOH | Product | MH⁺ |
| 3 | 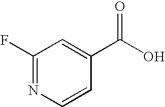 | 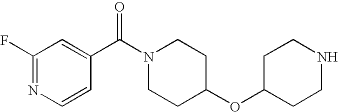 3A | 8%/308 |
| 4 | 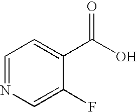 | 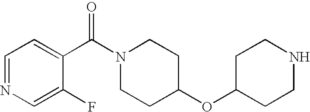 4A | 45%/308 |

### Example 5

### Preparation of Intermediate Compound 5B

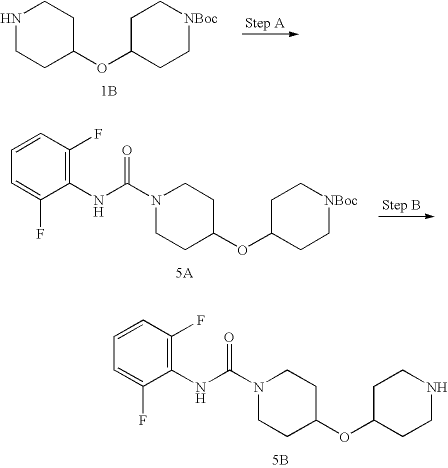

### Step A

Compound 1B (200 mg, 0.704 mmol) was dissolved in 10 mL of CH₂Cl₂. Triethylamine (213 mg, 2.112 mmol) was added followed by 2,5-difluorophenylisocyanate (328 mg, 2.112 mmol) and the resulting reaction was stirred at room temperature for 18 hours. The reaction mixture was then diluted with CH₂Cl₂, washed with a 1N NaOH aqueous solution, and the organic layer was dried over sodium sulfate and concentrated in vacuo. The residue obtained was purified using flash column chromatography (EtOAc/Hexanes, 1:2) to provide compound 5A as a yellow oil.

### Step B

A solution of compound 5A in trifluoroacetic acid (5 mL) and CH₂Cl₂ (5 mL) was allowed to stir for 30 minutes, then concentrated in vacuo. The residue obtained was dissolved in CH₂Cl₂ and 1N NaOH aqueous solution was added (5 mL). The resulting mixture was stirred for 10 minutes, then the organic layer was separated, dried over MgSO₄, filtered and concentrated in vacuo to provide compound 5B (70 mg, 30%).

### Example 6

### Preparation of Intermediate Compound 6B

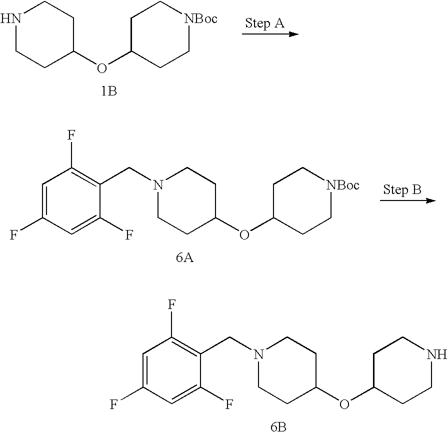

### Step A

Compound 1B (200 mg, 0.704 mmol) was dissolved in 10 mL of CH₂Cl₂. 2,4,6-trifluorobenzaldehyde (225 mg, 1.408 mmol) and acetic acid (0.1 mL) were then added and the reaction was allowed to stir for 10 minutes. Na(OAc)₃BH (313 mg, 1.408 mmol) was then added and the resulting mixture was allowed to stir at room temperature for 18 hours, then diluted with CH₂Cl₂, washed with a 1N NaOH aqueous solution and brine, dried over MgSO₄, filtered and concentrated in vacuo. The resulting residue was purified using flash column chromatography on silica gel (MeOH—CH₂Cl₂, 1:10) to provide compound 6A as a yellow oil.

### Step B

A solution of compound 6A in trifluoroacetic acid (5 mL) and CH₂Cl₂ (5 mL) was allowed to stir for 30 minutes, then concentrated in vacuo. The residue obtained was dissolved in CH₂Cl₂ and 1N NaOH aqueous solution was added. The resulting mixture was stirred for 10 minutes, then the organic layer was separated, dried over MgSO₄, filtered and concentrated in vacuo to provide compound 6B (60 mg, 26%).

### Example 7

### Preparation of Intermediate Compound 7B

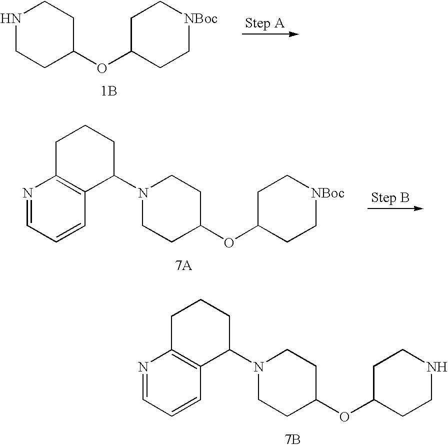

### Step A

5,6,7,8-Tetrahydroquinolin-5-ol (110 mg, 0.743 mmol) was dissolved in 6 mL of CH₂Cl₂ and the resulting solution was cooled to 0° C. Triethylamine (0.26 mL, 1.87 mmol) was then added followed by methanesulfonyl chloride (72 uL, 0.926 mmol) and the reaction was allowed to stir for about 2 hours at 0° C., then stirred at room temperature for an additional 2 hours. The reaction mixture was then added dropwise to a stirred mixture of compound 1B (200 mg, 0.703 mmol, available from Example 1 Step B) and triethylamine (0.3 mL, 2.15 mmol) in 5 mL of CH₂Cl₂. The reaction was heated to reflux and allowed to stir at this temperature for about 15 hours. The reaction mixture was then cooled to RT, diluted with CH₂Cl₂ and the organic layer was washed with H₂O and brine, dried over Na₂SO₄, and concentrated in vacuo to provide a crude yellow oil. The crude oil was purified using preparative TLC(CH₂Cl₂-MeOH=25:1, v/v) to provide 46 mg of compound 7A (15%) as a near colorless oil.

### Step B

A solution of compound 7A (46 mg, 0.111 mmol) in 2 mL of CH₂Cl₂ and 0.5 mL of trifluoroacetic was allowed to stir at RT for about 15 hours. A 1.0 M NaOH aqueous solution was then added to the reaction mixture and the resulting solution was extracted with CH₂Cl₂ (60 mL). The organic layer was washed with brine, dried over Na₂SO₄ and concentrated in vacuo to provide 33 mg of compound 7B as a yellow oil (MH⁺=316.25).

### Example 8

### Preparation of Intermediate Compound 8B

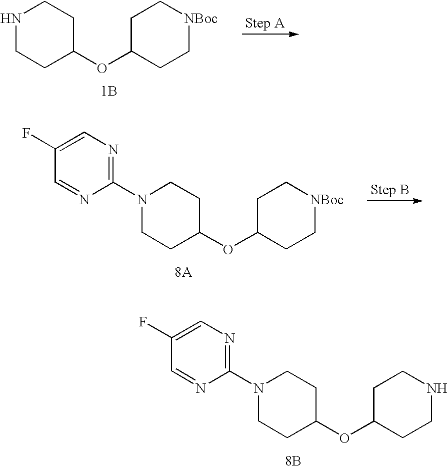

### Step A

To a stirred solution of compound 1B (270 mg, 0.95 mmol) in 2 mL of CH₂Cl₂ and 3 mL of toluene was added 2-chloro-5-fluoro-pyrimidine (0.1 g, 0.755 mmol), followed by diisopropylethylamine (0.2 mL, 1.148 mmol). The reaction was heated to 90° C. and allowed to stir at this temperature for about 48 hours. The reaction was then cooled to RT and concentrated in vacuo to provide an oily residue which was purified using preparative TLC (CH₂Cl₂-MeOH=50:1, v/v) to provide 56 mg of compound 8A (19.5%, MH⁺=381.2) as a near colorless oil which solidified on standing.

### Step B

Using the method described in Example 7 Step B, compound 8A was converted to compound 8B (46 mg, crude) as a yellow oil.

### Example 9

### Preparation of Intermediate Compound 9B

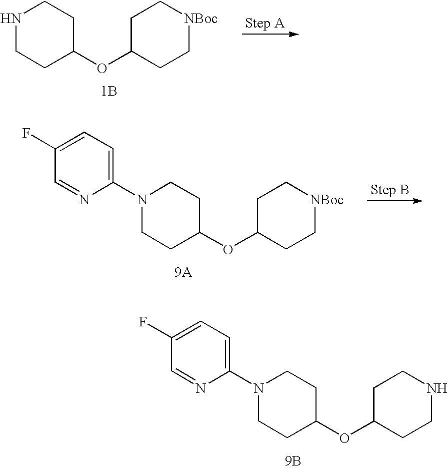

### Step A

To a solution of 2-bromo-5-fluoropyridine (259 mg, 2.84 mmol) and compound 1B (350 mg, 1.23 mmol) in toluene (15 mL) was added anhydrous NaO^{t}Bu (165 mg, 1.72 mmol), Pd(OAc)₂ (28 mg, 0.123 mmol) and 2-(di-t-butylphosphino)biphenyl (33 mg, 0.111 mmol). The reaction was heated to 100° C. and allowed to stir at this temperature for 4 hours under N₂ atmosphere. The reaction mixture was then cooled to room temperature, filtered through a pad of Celite® and concentrated in vacuo. The residue obtained was purified using flash column chromatography (EtOAc/Hexanes, 1:9, then 1:4) to provide compound 9A as a yellow oil.

### Step B

A solution of compound 9A in trifluoroacetic acid (5 mL) and CH₂Cl₂ (5 mL) was allowed to stir for 30 minutes, then concentrated in vacuo. The residue obtained was dissolved in CH₂Cl₂ and to the resulting solution was added a 1N NaOH aqueous solution. The mixture was stirred for 10 minutes, then the organic layer was separated, dried over MgSO₄, filtered, and concentrated in vacuo to provide compound 9B (160 mg, 47%).

### Example 10

### Preparation of Intermediate Compound 10B

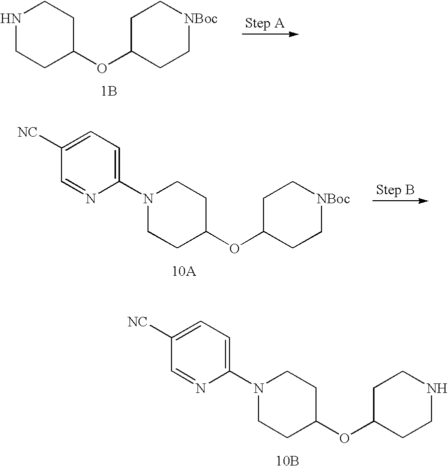

Using the method described in Example 9, compound 1B (450 mg, 1.58 mmol) was converted to compound 10B (170 mg, 38%).

### Example 11

### Preparation of Intermediate Compound 11B

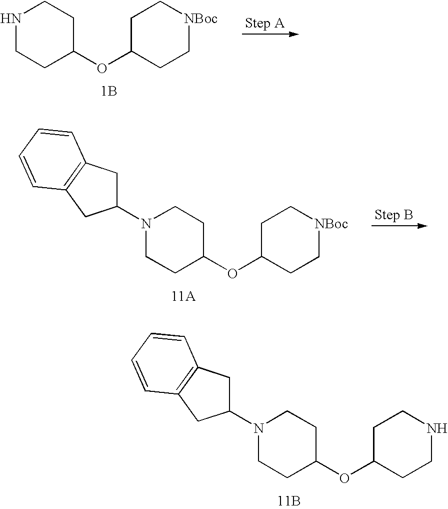

Using the method described in Example 1 Step E, compound 1B (288 mg, 1.01 mmol) was reacted with 2-indanone (57 mg, 0.43 mmol) to provide compound 11A. Removal of the Boc protecting group using the method described in Example 10, Step B, provided compound 11B (56 mg, 32%, MH⁺=301.27).

### Examples 12-23

### Preparation of Compounds 1, 5-7, 10-15, 23 and 24

Using the methods described in Example 1, Steps E and F, the piperidyl intermediates and aldehyde intermediates specified in the table below were reacted with each other to provide compounds 1, 5-7, 10-15, 23 and 24.

| Ex. | Piperidine | Aldehyde | Product | Yield/ |
|---|---|---|---|---|
| No. | Intermediate | Intermediate | (compound no.) | M + H |
| 12 | 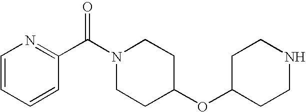 | 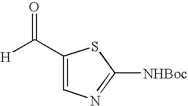 | 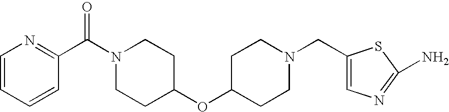 5 | 29% 402.2 |
| 13 | 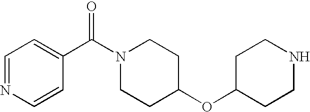 | 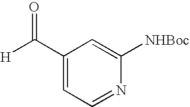 | 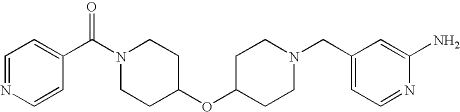 7 | 61.5% 396.2 |
| 14 | 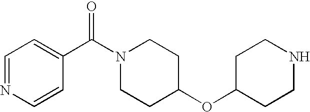 | 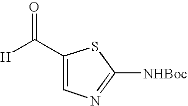 | 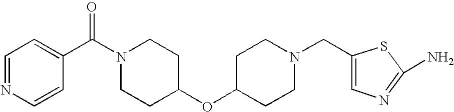 6 | 21% 402.2 |
| 15 | 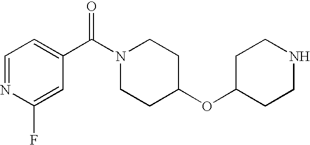 | 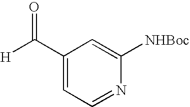 | 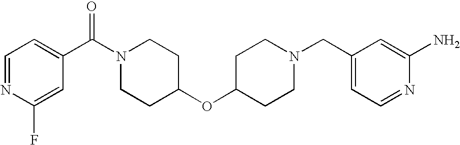 13 | 9% 414 |
| 16 | 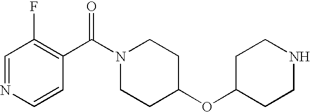 | 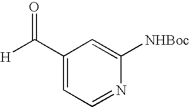 | 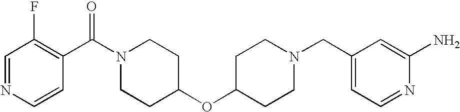 15 | 29% 414 |
| 17 | 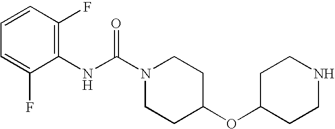 | 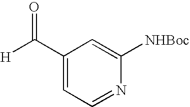 | 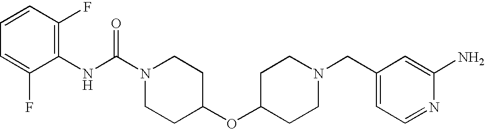 11 | 25% 446.2 |
| 18 | 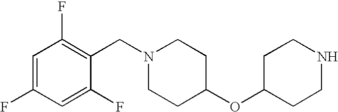 | 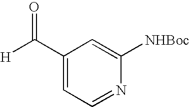 | 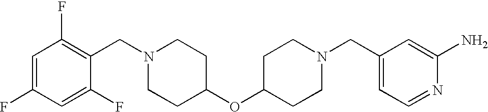 12 | 52% 435.2 |
| 19 | 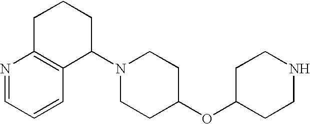 | 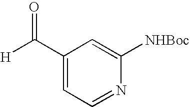 | 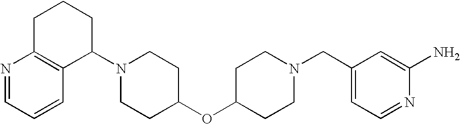 10 | 43.4% 422.2 |
| 20 | 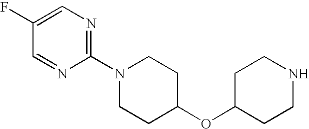 | 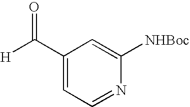 | 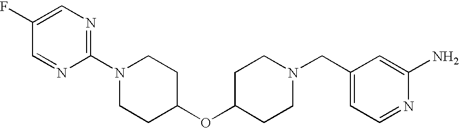 1 | 78% 387.1 |
| 21 | 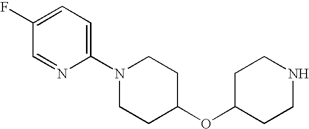 | 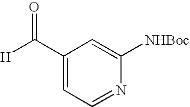 | 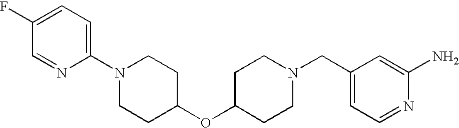 14 | 28% 386.2 |
| 22 | 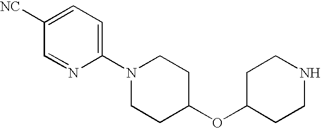 | 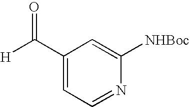 | 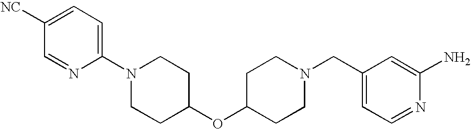 23 | 59% — |
| 23 | 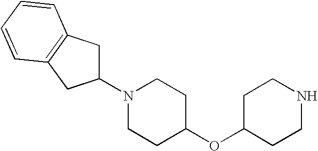 | 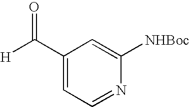 | 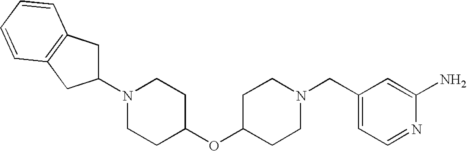 24 | 29% 407 |

### Example 24

### Preparation of Intermediate Compound 24B

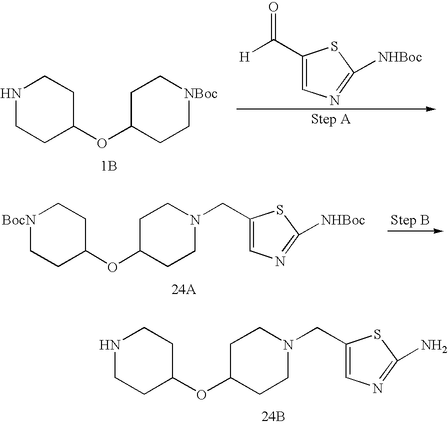

### Step A

To a stirred solution of compound 1B (284 mg, 1.0 mmol), in 6 mL of CH₂Cl₂ at room temperature was added N-boc-2-aminothiazole-4-carboxaldehyde (296 mg, 1.297 mmol). The mixture was then treated with sodium triacetoxyborohydride (275 mg, 1.298 mmol) and a catalytic amount of acetic acid. The reaction was allowed to stir for about 15 hours, then H₂O was added and the aqueous mixture was extracted with CH₂Cl₂. The organic extract was washed with brine, dried over Na₂SO₄, and concentrated in vacuo to provide a crude yellow solid. The crude solid was purified using preparative TLC(CH₂Cl₂-7N NH₃ in MeOH=30:1, v/v) to provide 271 mg of compound 24A (55%, MH⁺=497.22) as a light yellow solid.

### Step B

A solution of compound 24 (271 mg) in 5 mL CH₂Cl₂ and 1 mL of trifluoroacetic acid was stirred at room temperature for about 15 hours. A 1.0 M NaOH aqueous solution was then added (15 mL) and the resulting mixture was extracted with CH₂Cl₂ (60 mL, 20 mL) The organic extract was washed with brine, dried over Na₂SO₄, and concentrated in vacuo to provide 106 mg of compound 24B (66%, MH⁺=297.19) as a light yellow solid.

### Examples 25-27

### Preparation of Compounds 2, 3 and 8

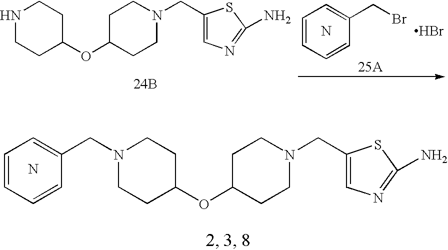

Compound 24B (1 mol. equiv.) was dissolved in 2 mL of methanol and to the resulting solution was added triethylamine (3 mol. equiv.) followed by a pyridinyl methyl bromide hydrobromide of formula 25A (1.1 mol. equiv.), as specified in the table below. The mixture was stirred for 3 to 5 hours, concentrated in vacuo, and the crude material obtained was partitioned between CH₂Cl₂ (50 mL) and a saturated aqueous NaHCO₃ solution (15 mL). The organic layer was washed with brine, dried over Na₂SO₄, concentrated in vacuo and the residue obtained was purified using preparative TLC(CH₂Cl₂-7N NH₃ in MeOH=25:1, v/v) to provide the corresponding products 2, 3 and 8 as shown in the table below.

| Example | | Product | |
|---|---|---|---|
| No. | 25A | (Compound No.) | Yield/MH⁺ |
| 25 | 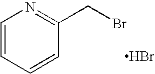 | 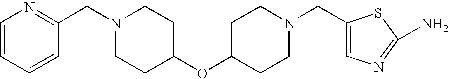 2 | 19%/388.2 |
| 26 | 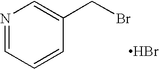 | 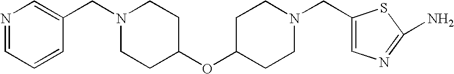 8 | 3.6%/388.2 |
| 27 | 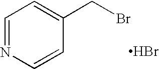 | 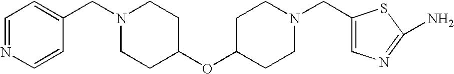 3 | 26%/388.2 |

### Example 28

### Preparation of Compound 17

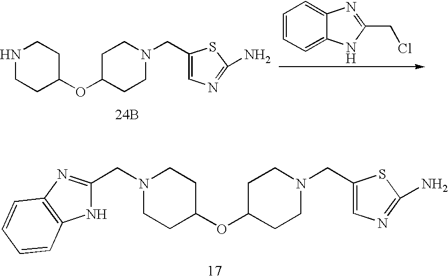

Using the method described in Examples 27-29, compound 24B (23 mg, 0.079 mmol, available from Example 24) was reacted with 2-chloromethylbenzimidazole (15 mg, 0.09 mmol) to provide compound 17 (3.8 mg, 11.2%, MH⁺=427.2) as a light yellow solid.

### Example 29

### Preparation of Compound 19

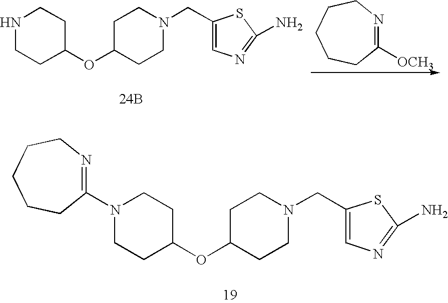

Compound 24B (23 mg, 0.079 mmol) and 1-aza-2-methoxy-1-cycloheptene were placed in a 2-dram vial. Toluene (1 mL) and ethanol (0.5 mL) were added, the vial was capped, and the reaction was heated to 85° C. and allowed to stir at this temperature for 12 hours. The reaction mixture was cooled to RT, then concentrated in vacuo and the oily residue obtained was purified using preparative TLC(CH₂Cl₂-7N NH₃ in MeOH=20:1, 10:1, v/v) to provide compound 19 (43%, MH⁺=392.17) as an viscous oil.

### Example 30

### Preparation of Compound 22

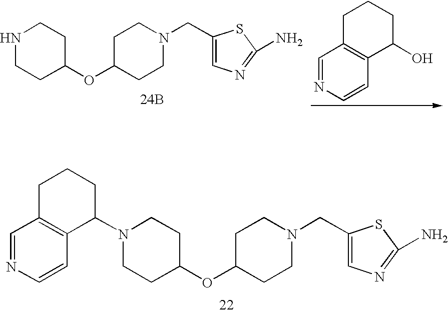

To a stirred solution of 5,6,7,8-tetrahydroisoquinolin-5-ol (42 mg, 0.282 mmol, obtained by NaBH4 reduction of the commercially available ketone precursor), in 2 mL of CH₂Cl₂ at room temperature was added triethylamine (90 μL, 0.646 mmol) followed by methanesulfonic anhydride (50 mg, 0.287 mmol). The reaction was allowed to stir for 1 hour, then a solution of compound 24B (55 mg, 0.186 mmol, available from Example 24) in 2 mL of CH₂Cl₂ was added. Additional triethylamine (50 μL, 0.359 mmol) was added and the reaction was allowed to stir for about 60 hours and was then concentrated in vacuo. The residue obtained was dissolved in CH₂Cl₂ (50 mL) and the organic layer was washed with H₂O and brine, dried over Na₂SO₄, and concentrated in vacuo to provide an oily residue. The residue was then purified using preparative TLC(CH₂Cl₂-7N NH₃ in MeOH=15:1, v/v) to provide 6.8 mg of compound 22 (9%, MH⁺=428.2) as a yellow solid.

### Example 31

### Preparation of Intermediate Compound 31B

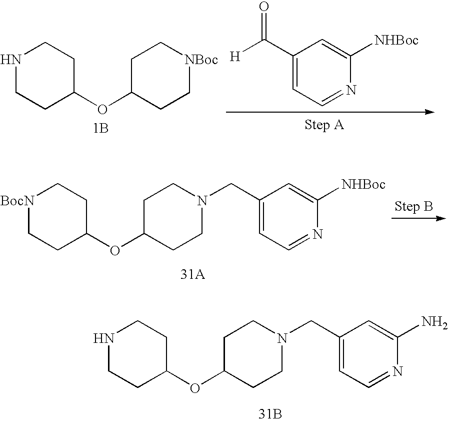

Using the method described in Example 24, compound 1B (248 mg, 0.87 mmol), was converted to compound 31B (118 mg, 47% over two steps, MH⁺=291.24) as a yellow solid.

### Examples 32-33

### Preparation of Compounds 9 and 18

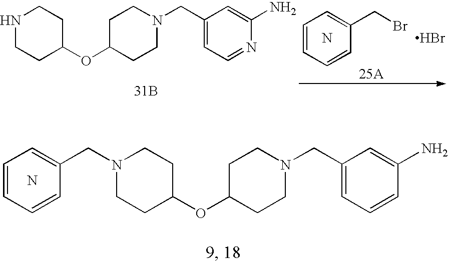

Using the method described in Examples 25-27, compound 31B was reacted with a pyridinyl methyl bromide hydrobromide of formula 25A as specified in the table below to provide compounds 9 and 18.

| Example | | | |
|---|---|---|---|
| No. | 25A | Product | Yield/MH⁺ |
| 32 | 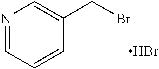 | 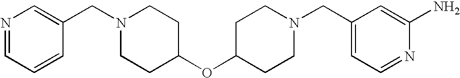 9 | 17%/382 |
| 33 | 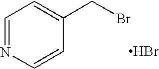 | 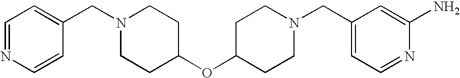 18 | 65%/382.2 |

### Example 34

### Preparation of Compound 21

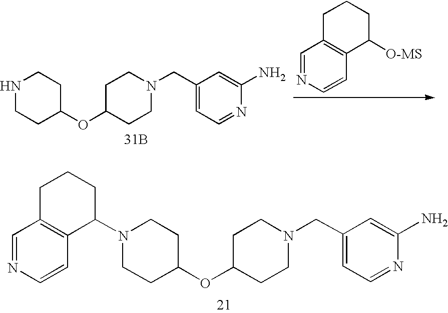

Using the method describe in Example 30, compound 31B (40 mg, 0.138 mmol) was reacted with the mesylate of 5,6,7,8-tetrahydroisoquinolin-5-ol (30 mg, 0.201 mmol) to provide compound 21 (8%, MH⁺=422.2) as a light yellow solid.

### Example 35

### Preparation of Intermediate Compound 30

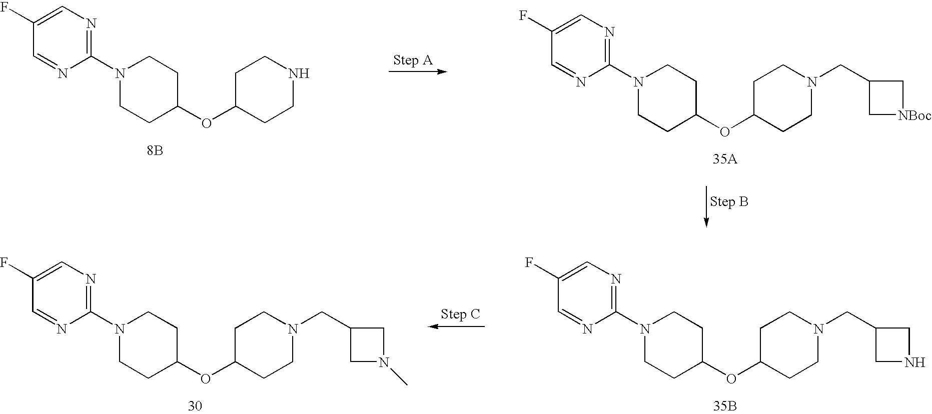

### Step A

Compound 8B (105 mg, 0.37 mmol), available from Example 8, was reacted with 1-boc-azetidine-3-formaldehyde (85 mg, 0.46 mmol) using the method described in Example 1 step E to provide intermediate compound 35A (130 mg, 78%, MH⁺=450.13).

### Step B

Compound 35A (130 mg, 0.29 mmol) was dissolved in 4 mL of CH₂Cl₂ and the resulting solution was cooled to −78° C. Iodotrimethylsilane (0.12 mL, 0.87 mmol) was added and the reaction was allowed to stir for 4 hours, during which time, the reaction was slowly allowed to warm to 0° C. A 1.0 M NaOH aqueous solution (20 mL) was added to the reaction mixture and the resulting aqueous mixture was extracted with CH₂Cl₂. The organic extract was washed with brine, dried over Na₂SO₄, filtered, and concentrated in vacuo to provide 127 mg of compound 35B (crude, MH⁺=350.17).

### Step C

Compound 35B (127 mg) was dissolved in 5 mL of CH₂Cl₂, and to the resulting solution was added an aqueous formaldehyde solution (37% in H₂O, 0.2 mL, 0.87 mmol), followed by sodium triacetoxyborohydride (184 mg, 0.87 mmol) and a catalytic amount of acetic acid. The reaction was allowed to stir for about 60 hours, then a saturated aqueous. NaHCO₃ solution was added. The aqueous mixture was extracted with CH₂Cl₂ and the organic extract was washed with brine, dried by Na₂SO₄, and concentrated in vacuo to provide a crude oil. The crude oil was purified using preparative TLC eluting with CH₂Cl₂-7N NH₃ in MeOH (92:8, v/v) to provide 48 mg of compound 30 (46%, MH⁺=364.2) as a yellow oily solid.

### Example 36

### Preparation of Compound 28

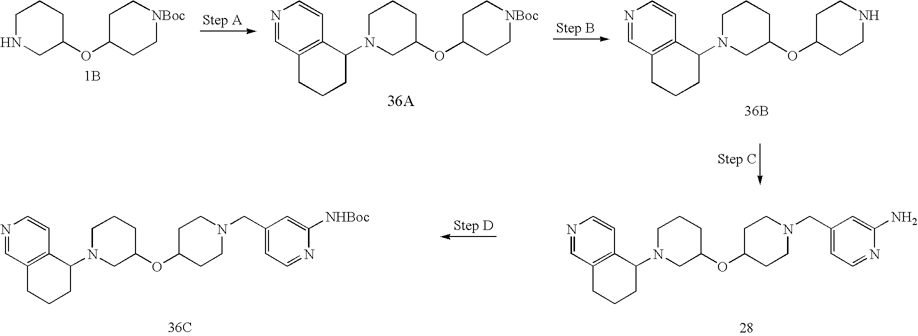

### Step A

To a stirred first solution of 5,6,7,8-tetrahydroisoquinolin-5-ol (165 mg, 1.106 mmol) in 5 mL of CH₂Cl₂ was added triethylamine (0.25 mL, 1.794 mmol) and methanesulfonyl chloride (0.14 mL, 1.182 mmol). The reaction was allowed to stir at room temperature for 45 minutes. In a separate flask, sodium hydride (45 mg, 1.125 mmol, 60% in mineral oil) was added to a solution of compound 1B (292 mg, 1.026 mmol) in 5 mL of DMF. This second solution was stirred for 30 minutes, then added to the first solution and the resulting reaction was allowed to stir for about 36 hours. Water was then added to the reaction mixture and the resulting aqueous mixture was extracted with CH₂Cl₂ (50 mL×3). The combined organic extracts were washed with brine, dried over Na₂SO₄, and concentrated in vacuo to provide an oily residue which was purified using preparative TLC (CH₂Cl₂-7N NH₃ in MeOH=30:1 v/v) to provide 128 mg of compound 36A (30%, MH⁺=416.2).

### Step B

Using the method described in Example 24, Step B, compound 36A (126 mg, 0.303 mmol) was converted to compound 36B (88.5 mg, 92%, MH⁺=316.22).

### Steps C & D

Following the procedures described in Example 1, Steps E and F, t compound 36B (88 mg, 0.278 mmol) was coupled with N-Boc-2-amino-4-formal pyridine (83 mg, 0.374 mmol) to provide intermediate 36C, which upon further deprotection was converted to compound 28 (61 mg, 52%, MH⁺=422.2).

### Example 37

### Preparation of Compound 27

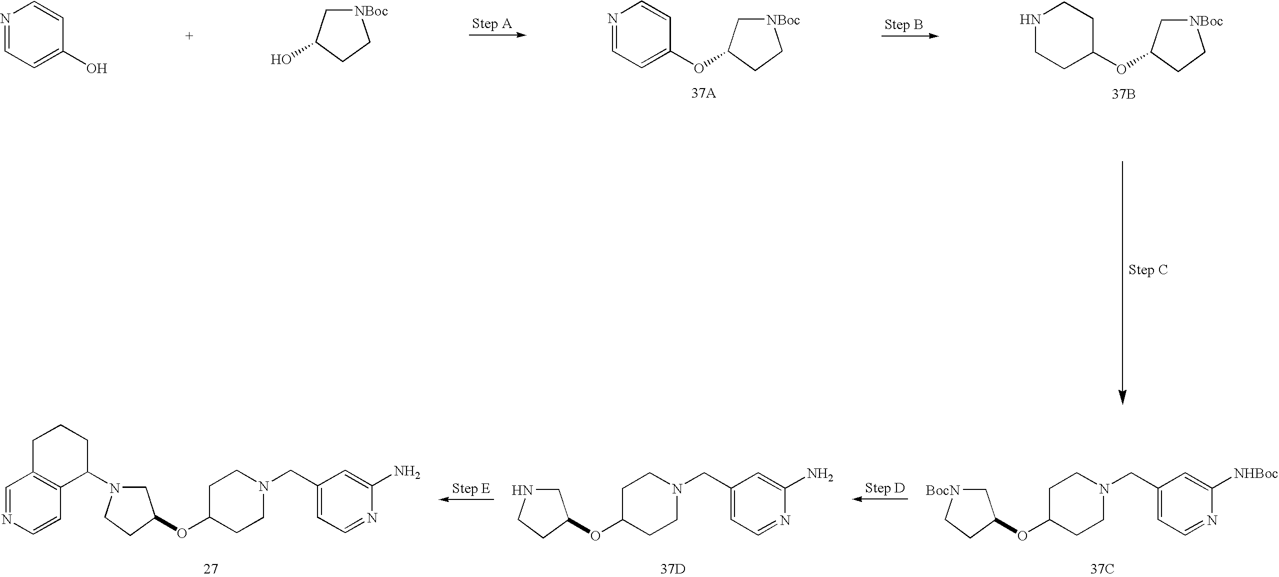

### Step A

Using the method described in Example 1, Step A, 4-hydroxypyridine (1.0 g, 10.51 mmol) was coupled with (S)-1-Boc-3-hydroxypyrrolidine (2.46 g, 13.14 mmol) to provide compound 37A (1.98 g, 71%, MH⁺=265.1).

### Step B

Using the method described in Example 1, Step B, compound 37A (1 g, 3.78 mmol) was converted to compound 37B (0.786 g, 77%, MH⁺=271.1).

### Step C

Using the method described in Example 24, Step A, compound 37B (256 mg, 0.947 mmol) was reacted with N-boc-2-amino-4-formal pyridine (273 mg, 1.23 mmol) to provide intermediate 37C (339 mg, 75%, MH⁺=477.24).

### Step D

Using the method described in Example 24, Step B, compound 37C (0.33 g, 0.69 mmol) was converted to compound 37D (74%, MH⁺=277.17).

### Step E

Using the method described in Example 30, compound 37D (140 mg, 0.53 mmol), was coupled with the mesylate of 5,6,7,8-tetrahydroisoquinolin-5-ol (52 mg, 0.35 mmol) to provide compound 27 (33 mg, 24%, MH⁺=408.2).

### Example 38

### Preparation of Compound 26

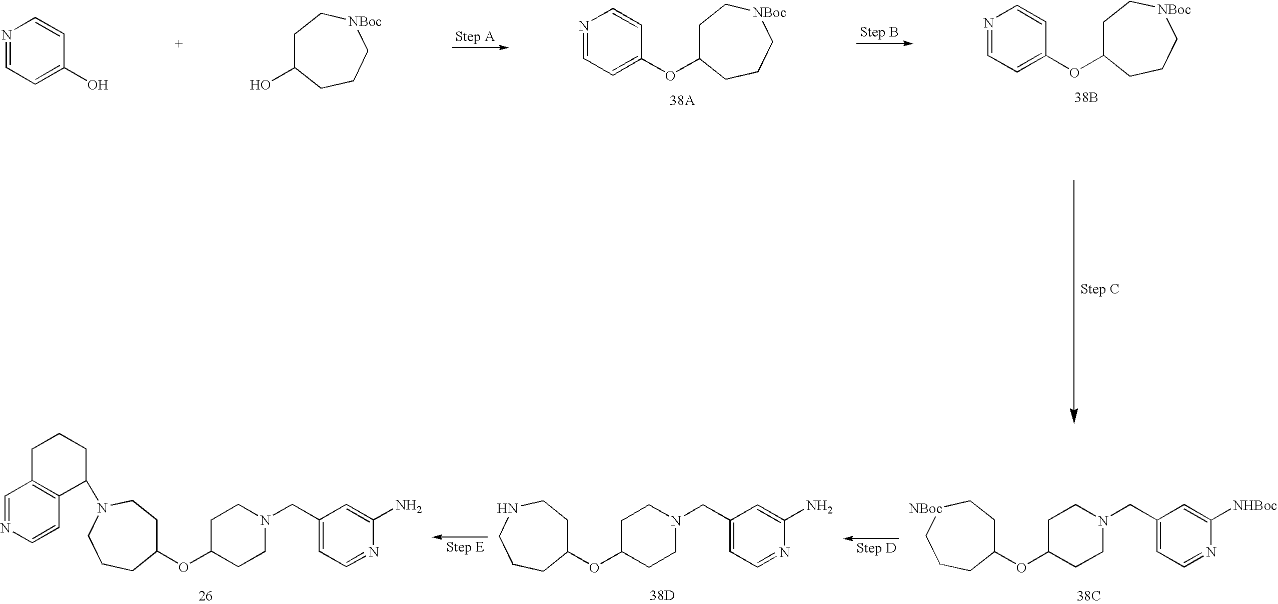

### Step A

Using the method described in Example 1, Step A, 4-hydroxypyridine (1.0 g, 10.51 mmol) was coupled with Boc-4-hydroxy-azepane (2.83 g, 13.145 mmol) to provide compound 38A (2.13 g, 69%, MH⁺=293.2).

### Step B

Using the method described in Example 1, Step B, compound 38A (1 g, 3.42 mmol) was converted to compound 38B (0.34 g, 34%, MH⁺=299.24).

### Step C

Using the method described in Example 24, Step A, compound 38B (244 mg, 0.814 mmol) was reacted with N-boc-2-amino-4-formal pyridine (273 mg, 1.23 mmol) to provide compound 38C (345 mg, 84%, MH⁺=505.30).

### Step D

Using the method described in Example 24, Step B, compound 38C (340 mg, 0.674 mmol) was converted to compound 38D (160 mg, 80%, MH⁺=305.18).

### Step E

Using the method described in Example 30, compound 38D (160 mg, 0.53 mmol) was coupled with the mesylate of 5,6,7,8-tetrahydroisoquinolin-5-ol (52 mg, 0.35 mmol) to provide compound 26 (10 mg, 7%, MH⁺=436.2).

### Example 39

### Preparation of Compound 29

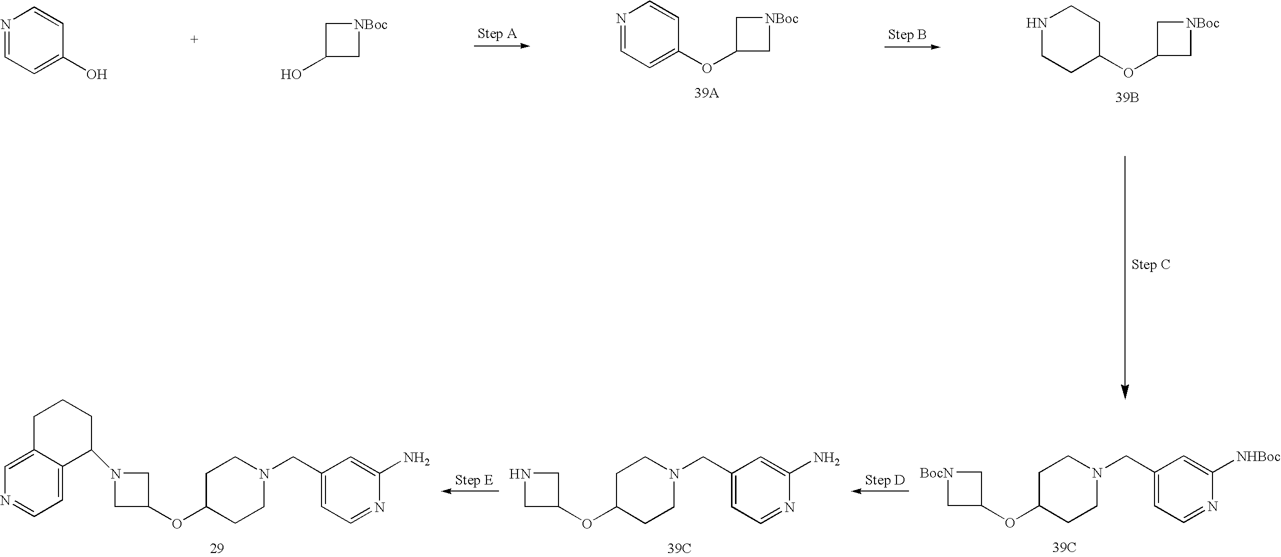

### Step A

Using the method described in Example 1, Step A, 4-hydroxypyridine (2.0 g, 21.03 mmol) was coupled with 1-boc-3-hydroxy-azetidine (4.55 g, 26.27 mmol) to provide compound 39A (4.10 g, 78%, MH⁺=251.1).

### Step B

Using the method described in Example 1, Step B, compound 39A (1 g, 3.78 mmol) was converted to compound 39B (0.62 g, 61%, MH⁺=257.23).

Steps C through E

Following the procedures set forth in Example 37, Steps C to E, compound 39B was converted to compound 29.

### Example 40

### Preparation of Compound 20

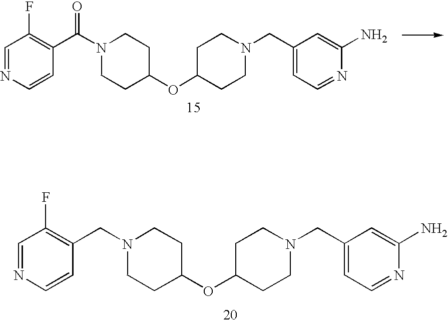

Compound 15 (42 mg, 0.10 mmol) was dissolved in 2 mL of THF and cooled to 0° C. A 2.0 M solution of boron-methyl sulfide complex in THF was added and the resulting reaction was allowed to stir at 0° C. for 30 minutes, then heated to reflux and allowed to stir at this temperature for 30 minutes. The reaction mixture was then cooled to 0° C., stirred a this temperature for 30 minutes, then heated to reflux and allowed to stir at this temperature for 30 minutes. The reaction mixture was once again re-cooled to 0° C. and a 6 M HCl aqueous solution was added (0.1 mL). The resulting solution was heated to reflux and then concentrated in vacuo. The solid residue obtained was treated with 1 mL of a 4 M aqueous NaOH solution, then potassium carbonate was added until the solution became saturated. The resulting saturated solution was extracted with ethyl acetate and the organic extract was filtered through a thin layer of potassium carbonate in a fritted funnel and the filtrate was concentrated in vacuo. The oily residue obtained was purified using preparative TLC with CH₂Cl₂-7N NH₃ in MeOH (97:3, v/v) to provide 28 mg of compound 20 (51%, MH⁺=400.2).

### Example 41

### Preparation of Compound 16

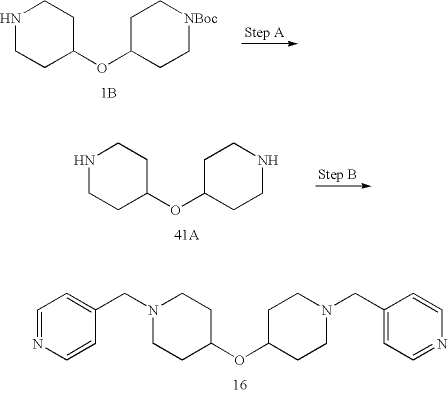

### Step A

Using the method described in Example 7 Step B, compound 1B (96 mg, 0.34 mmol) was converted to compound 41A (140 mg, crude)

### Step B

Using the method described in Example 27, compound 41A (crude material 140 mg), was alkylated with 4-bromomethylpyridine hydrobromide salt (196 mg, 0.78 mmol) to provide compound 16 (16 mg, 13% over two steps, MH⁺=367.2).

### Example 42

### H₃ Receptor Binding Assay

The source of H₃ receptors was recombinant human receptor, expressed in HEK-293 (human embryonic kidney) cells.

All illustrative Oxypiperidine Derivatives of the present invention to be tested were dissolved in DMSO and then diluted into the binding buffer (50 mM Tris, pH 7.5) such that the final concentration was 2 μg/ml with 0.1% DMSO. Membranes were then added (5 μg in the case of recombinant human receptor) to the reaction tubes. The reaction was initiated by the addition of 3 nM [³H]R-α-methyl histamine (8.8 Ci/mmol) or 3 nM [³H]N^{α}-methyl histamine (80 Ci/mmol) and continued under incubation at 30° C. for 30 minutes. Bound ligand was separated from unbound ligand by filtration, and the amount of radioactive ligand bound to the membranes was quantitated using liquid scintillation spectrometry. All incubations were performed in duplicate and the standard error was always less than 10%. Compounds that inhibited more than 70% of the specific binding of radioactive ligand to the receptor were serially diluted to determine a Kᵢ (nM).

Selected Oxypiperidine Derivatives of the present invention demonstrated Kᵢ values within the range of about 1 nM to about 10 μM when tested in this assay.

### Example 43

### In Vivo Effect of Compounds of the Invention on Glucose Levels in Diabetic Mice

Five-week-old male ICR mice are used in this assay (and can be purchased, for example, from Taconic Farm (Germantown, N.Y.)) are to be placed on a “western diet” containing 45% (kcal) fat from lard and 0.12% (w/w) cholesterol. After 3 weeks of feeding, the mice are injected once with low dose streptozocin (STZ, ip 75-100 mg/kg) to induce partial insulin deficiency. Two weeks after receiving the STZ injection, the STZ-treated mice are the evaluated and those that have developed type 2 diabetes and display hyperglycemia, insulin resistance, and glucose intolerance are selected and placed in one of three groups: (1) a non-treated control group, (2) a group treated with rosiglitazone (5 mg/kg/day in diet) for four weeks; and (3) a group treated with an Oxypiperidine Derivative of the present invention (10/mg/kg in diet) for four weeks. After 4 weeks, the mice in each group are evaluated for glucose levels and the treatment group can then be compared to the rosiglitazone group and to the control group in order to evaluate the effect of the test compound.

### Example 44

### In Vivo Effect of Compounds of the Invention on Glucose Levels in Diabetic Rats

Adult, diabetic, Goto-Kakizaki rats (14 weeks old) are used and are first tested for non-fasting glucose levels using a glucometer. Rats with glucose levels between 130 and 370 mg/dl are then randomized into treatment (N=10) and control (N=10) groups. Animals in the treatment group are administered an Oxypiperidine Derivative of the present invention in their food chow at a dose of 10 mg/kg/day. After one week of treatment, blood can be collected via tail snip and the non-fasting glucose level can be measured using a glucometer.

### Uses of the Oxypiperidine Derivatives

The Oxypiperidine Derivatives are useful for treating or preventing a Condition a patient. Accordingly, the present invention provides methods for treating or preventing a Condition in patient, comprising administering to the patient an effective amount of one or more compounds of formula (I).

### Methods for Treating or Preventing Pain

The Oxypiperidine Derivatives are useful for treating or preventing pain in a patient.

Accordingly, in one embodiment, the present invention provides a method for treating pain in a patient, comprising administering to the patient an effective amount of one or more Oxypiperidine Derivatives.

Illustrative examples of pain treatable or preventable using the present methods, include, but are not limited to acute pain, chronic pain, neuropathic pain, nociceptive pain, cutaneous pain, somatic pain, visceral pain, phantom limb pain, cancer pain (including breakthrough pain), pain caused by drug therapy (such as cancer chemotherapy), headache (including migraine, tension headache, cluster headache, pain caused by arthritis, pain caused by injury, toothache, or pain caused by a medical procedure (such as surgery, physical therapy or radiation therapy).

In one embodiment, the pain is neuropathic pain.

In another embodiment, the pain is cancer pain.

In another embodiment, the pain is headache.

### Methods for Treating or Preventing Diabetes

The Oxypiperidine Derivatives are useful for treating or preventing diabetes in a patient. Accordingly, in one embodiment, the present invention provides a method for treating diabetes in a patient, comprising administering to the patient an effective amount of one or more Oxypiperidine Derivatives.

Examples of diabetes treatable or preventable using the Oxypiperidine Derivatives include, but are not limited to, type I diabetes (insulin-dependent diabetes mellitus), type II diabetes (non-insulin dependent diabetes mellitus), gestational diabetes, autoimmune diabetes, insulinopathies, idiopathic type I diabetes (Type Ib), latent autoimmune diabetes in adults, early-onset type 2 diabetes (EOD), youth-onset atypical diabetes (YOAD), maturity onset diabetes of the young (MODY), malnutrition-related diabetes, diabetes due to pancreatic disease, diabetes associated with other endocrine diseases (such as Cushing's Syndrome, acromegaly, pheochromocytoma, glucagonoma, primary aldosteronism or somatostatinoma), type A insulin resistance syndrome, type B insulin resistance syndrome, lipatrophic diabetes, diabetes induced by β-cell toxins, and diabetes induced by drug therapy (such as diabetes induced by antipsychotic agents).

In one embodiment, the diabetes is type I diabetes.

In another embodiment, the diabetes is type II diabetes.

### Methods for Treating or Preventing a Diabetic Complication

The Oxypiperidine Derivatives are useful for treating or preventing a diabetic complication in a patient. Accordingly, in one embodiment, the present invention provides a method for treating a diabetic complication in a patient, comprising administering to the patient an effective amount of one or more Oxypiperidine Derivatives.

Examples of diabetic complications treatable or preventable using the Oxypiperidine Derivatives include, but are not limited to, diabetic cataract, glaucoma, retinopathy, aneuropathy (such as diabetic neuropathy, polyneuropathy, mononeuropathy, autonomic neuropathy, microaluminuria and progressive diabetic neuropathyl), nephropathy, gangrene of the feet, immune-complex vasculitis, systemic lupus erythematosus (SLE), atherosclerotic coronary arterial disease, peripheral arterial disease, nonketotic hyperglycemic-hyperosmolar coma, foot ulcers, joint problems, a skin or mucous membrane complication (such as an infection, a shin spot, a candidal infection or necrobiosis lipoidica diabeticorumobesity), hyperlipidemia, hypertension, syndrome of insulin resistance, coronary artery disease, a fungal infection, a bacterial infection, and cardiomyopathy.

### Methods for Treating or Preventing Obesity

The Oxypiperidine Derivatives are useful for treating or preventing obesity in a patient. Accordingly, in one embodiment, the present invention provides a method for treating obesity in a patient, comprising administering to the patient an effective amount of one or more Oxypiperidine Derivatives.

### Methods for Treating or Preventing Impaired Glucose Tolerance

The Oxypiperidine Derivatives are useful for treating or preventing impaired glucose tolerance in a patient.

Accordingly, in one embodiment, the present invention provides a method for treating impaired glucose tolerance in a patient, comprising administering to the patient an effective amount of one or more Oxypiperidine Derivatives.

### Methods for Treating or Preventing Impaired Fasting Glucose

The Oxypiperidine Derivatives are useful for treating or preventing impaired fasting glucose in a patient.

Accordingly, in one embodiment, the present invention provides a method for treating impaired fasting glucose in a patient, comprising administering to the patient an effective amount of one or more Oxypiperidine Derivatives.

### Methods for Treating a Cardiovascular Disease

The Oxypiperidine Derivatives are useful for treating or preventing a cardiovascular disease in a patient.

Accordingly, in one embodiment, the present invention provides a method for treating a cardiovascular disease in a patient, comprising administering to the patient an effective amount of one or more Oxypiperidine Derivatives.

Illustrative examples of cardiovascular diseases treatable or preventable using the present methods, include, but are not limited to atherosclerosis, congestive heart failure, cardiac arrhythmia, myocardial infarction, atrial fibrillation, atrial flutter, circulatory shock, left ventricular hypertrophy, ventricular tachycardia, supraventricular tachycardia, coronary artery disease, angina, infective endocarditis, non-infective endocarditis, cardiomyopathy, peripheral artery disease, Reynaud's phenomenon, deep venous thrombosis, aortic stenosis, mitral stenosis, pulmonic stenosis and tricuspid stenosis.

In one embodiment, the cardiovascular disease is atherosclerosis.

In another embodiment, the cardiovascular disease is congestive heart failure.

In another embodiment, the cardiovascular disease is coronary artery disease.

### Methods for Treating a Gastrointestinal Disorder

The Oxypiperidine Derivatives are useful for treating or preventing a gastrointestinal disorder in a patient.

Accordingly, in one embodiment, the present invention provides a method for treating a gastrointestinal disorder in a patient, comprising administering to the patient an effective amount of one or more Oxypiperidine Derivatives.

Illustrative examples of gastrointestinal disorders treatable or preventable using the present methods, include, but are not limited to gastroesophageal reflux disease (GERD), a gas-related complaint, a disorder related to hypermotility of the gastro-intestinal tract, a disorder related to hypomotility of the gastro-intestinal tract, chronic diarrhea, inflammatory bowel disease, Crohn's disease, ulcerative colitis, irritable bowel syndrome, dyspepsia, Celiac disease, pancreatitis, diverticulitis, gastritis, carbohydrate intolerance, dysphagia and Mallory-Weiss syndrome.

In one embodiment, the gastrointestinal disorder is GERD.

In another embodiment, the gastrointestinal disorder is a disorder related to hypermotility of the gastro-intestinal tract.

In another embodiment, the gastrointestinal disorder is a disorder related to hypomotility of the gastro-intestinal tract.

### Methods for Treating a CNS Disorder

The Oxypiperidine Derivatives are useful for treating or preventing a central nervous (CNS) system disorder in a patient.

Accordingly, in one embodiment, the present invention provides a method for treating a CNS disorder in a patient, comprising administering to the patient an effective amount of one or more Oxypiperidine Derivatives.

Illustrative examples of CNS disorders treatable or preventable using the present methods, include, but are not limited to hypoactivity of the central nervous system, hyperactivity of the central nervous system, a neurodegenerative disease, Alzheimer's disease, ALS, Creutzfeldt-Jakob disease, Huntington disease, multiple sclerosis, Lewy body disorder, a tic disorder, Tourette's Syndrome, Parkinson disease, Pick's disease, a prion disease or schizophrenia, epilepsy, migraine, anxiety, bipolar disorder, depression, attention deficit hyperactivity disorder (ADHD) and dementia.

In one embodiment, the CNS disorder is ADHD.

In another embodiment, the CNS disorder is hypoactivity of the central nervous system.

In another embodiment, the CNS disorder is hyperactivity of the central nervous system.

In still another embodiment, the CNS disorder is Alzheimer's disease.

In yet another embodiment, the CNS disorder is depression.

### Methods for Treating a Sleep Disorder

The Oxypiperidine Derivatives are useful for treating or preventing a sleep disorder in a patient.

Accordingly, in one embodiment, the present invention provides a method for treating a sleep disorder in a patient, comprising administering to the patient an effective amount of one or more Oxypiperidine Derivatives.

Illustrative examples of sleep disorders treatable or preventable using the present methods, include, but are not limited to insomnia, restless leg syndrome, bruxism, delayed sleep phase syndrome, hypopnea syndrome, narcolepsy, a parasomnia or sleep apnea.

In one embodiment, the sleep disorder is insomnia.

In another embodiment, the sleep disorder is restless leg syndrome.

### Methods for Treating Allergy

The Oxypiperidine Derivatives are useful for treating or preventing allergy in a patient.

Accordingly, in one embodiment, the present invention provides a method for treating allergy in a patient, comprising administering to the patient an effective amount of one or more Oxypiperidine Derivatives.

### Methods for Treating Allergy-Induced Airway Response

The Oxypiperidine Derivatives are useful for treating or preventing allergy-induced airway response in a patient.

Accordingly, in one embodiment, the present invention provides a method for treating allergy-induced airway responses in a patient, comprising administering to the patient an effective amount of one or more Oxypiperidine Derivatives.

### Methods for Treating Hypotension

The Oxypiperidine Derivatives are useful for treating or preventing hypotension in a patient.

Accordingly, in one embodiment, the present invention provides a method for treating hypotension in a patient, comprising administering to the patient an effective amount of one or more Oxypiperidine Derivatives.

### Combination Therapy

In one embodiment, the present invention provides methods for treating a Condition in a patient, the method comprising administering to the patient one or more Oxypiperidine Derivatives, or a pharmaceutically acceptable salt, solvate, ester or prodrug thereof and at least one additional therapeutic agent that is not an Oxypiperidine Derivative, wherein the amounts administered are together effective to treat or prevent a Condition.

Additional therapeutic agents useful in the present methods include, but are not limited to, an antiobesity agent, an antidiabetic agent, an agent useful for treating a cardiovascular disease, an agent useful for treating a gastrointestinal disorder, an agent useful for treating allergy or an allergy-induced airway response, an agent useful for treating congestion, an agent useful for treating a CNS disorder, an agent useful for treating hypotension, an analgesic agent, an agent useful for treating a sleep disorder, or any combination of two or more of these agents.

In another embodiment, the other therapeutic agent is an agent useful for reducing any potential side effect of an Oxypiperidine Derivative. Such potential side effects include, but are not limited to, nausea, vomiting, headache, fever, lethargy, muscle aches, diarrhea, general pain, and pain at an injection site.

In one embodiment, the additional therapeutic agent is an antidiabetic agent.

Non-limiting examples of antidiabetic agents useful in the present methods for treating a Condition include insulin sensitizers, alpha-glucosidase inhibitors, DPP-IV inhibitors, insulin secretagogues, hepatic glucose output lowering compounds, antihypertensive agents, sodium glucose uptake transporter 2 (SGLT-2) inhibitors, insulin and insulin-containing compositions, and anti-obesity agents as set forth above.

In one embodiment, the antidiabetic agent is an insulin secretagogue. In one embodiment, the insulin secretagogue is a sulfonylurea.

Non-limiting examples of sulfonylureas useful in the present methods include glipizide, tolbutamide, glyburide, glimepiride, chlorpropamide, acetohexamide, gliamilide, gliclazide, gliquidone, glibenclamide and tolazamide.

In another embodiment, the insulin secretagogue is a meglitinide.

Non-limiting examples of meglitinides useful in the present methods for treating a Condition include repaglinide, mitiglinide, and nateglinide.

In still another embodiment, the insulin secretagogue is GLP-1 or a GLP-1 mimetic.

Non-limiting examples of GLP-1 mimetics useful in the present methods include Byetta-Exanatide, Liraglutinide, CJC-1131 (ConjuChem, Exanatide-LAR (Amylin), BIM-51077 (Ipsen/LaRoche), ZP-10 (Zealand Pharmaceuticals), and compounds disclosed in International Publication No. WO 00/07617.

Other non-limiting examples of insulin secretagogues useful in the present methods include exendin, GIP and secretin.

In another embodiment, the antidiabetic agent is an insulin sensitizer.

Non-limiting examples of insulin sensitizers useful in the present methods include PPAR activators or agonists, such as troglitazone, rosiglitazone, pioglitazone and englitazone; biguanidines such as metformin and phenformin; PTP-1B inhibitors; and glucokinase activators.

In another embodiment, the antidiabetic agent is an alpha-glucosidase inhibitor.

Non-limiting examples of alpha-glucosidase inhibitors useful the present methods include miglitol, acarbose, and voglibose.

In another embodiment, the antidiabetic agent is an hepatic glucose output lowering agent.

Non-limiting examples of hepatic glucose output lowering agents useful in the present methods include Glucophage and Glucophage XR.

In yet another embodiment, the antidiabetic agent is insulin, including all formulations of insulin, such as long acting and short acting forms of insulin.

Non-limiting examples of orally administrable insulin and insulin containing compositions include AL-401 from AutoImmune, and the compositions disclosed in U.S. Pat. Nos. 4,579,730; 4,849,405; 4,963,526; 5,642,868; 5,763,396; 5,824,638; 5,843,866; 6,153,632; 6,191,105; and International Publication No. WO 85/05029, each of which is incorporated herein by reference.

In another embodiment, the antidiabetic agent is a DPP-IV inhibitor.

Non-limiting examples of DPP-IV inhibitors useful in the present methods include sitagliptin, saxagliptin (Januvia™, Merck), denagliptin, vildagliptin (Galvus™, Novartis), alogliptin, alogliptin benzoate, ABT-279 and ABT-341 (Abbott), ALS-2-0426 (Alantos), ARI-2243 (Arisaph), BI-A and BI-B (Boehringer Ingelheim), SYR-322 (Takeda), MP-513 (Mitsubishi), DP-893 (Pfizer), RO-0730699 (Roche) or a combination of sitagliptin/metformin HCl (Janumet™, Merck).

In a further embodiment, the antidiabetic agent is a SGLT-2 inhibitor.

Non-limiting examples of SGLT-2 inhibitors useful in the present methods include dapagliflozin and sergliflozin, AVE2268 (Sanofi-Aventis) and T-1095 (Tanabe Seiyaku).

Non-limiting examples of antihypertensive agents useful in the present methods for treating a Condition include β-blockers and calcium channel blockers (for example diltiazem, verapamil, nifedipine, amlopidine, and mybefradil), ACE inhibitors (for example captopril, lisinopril, enalapril, spirapril, ceranopril, zefenopril, fosinopril, cilazopril, and quinapril), AT-1 receptor antagonists (for example losartan, irbesartan, and valsartan), renin inhibitors and endothelin receptor antagonists (for example sitaxsentan).

In one embodiment, the antidiabetic agent is an agent that slows or blocks the breakdown of starches and certain sugars.

Non-limiting examples of antidiabetic agents that slow or block the breakdown of starches and certain sugars and are suitable for use in the compositions and methods of the present invention include alpha-glucosidase inhibitors and certain peptides for increasing insulin production. Alpha-glucosidase inhibitors help the body to lower blood sugar by delaying the digestion of ingested carbohydrates, thereby resulting in a smaller rise in blood glucose concentration following meals. Non-limiting examples of suitable alpha-glucosidase inhibitors include acarbose; miglitol; camiglibose; certain polyamines as disclosed in WO 01/47528 (incorporated herein by reference); voglibose. Non-limiting examples of suitable peptides for increasing insulin production including amlintide (CAS Reg. No. 122384-88-7 from Amylin; pramlintide, exendin, certain compounds having Glucagon-like peptide-1 (GLP-1) agonistic activity as disclosed in International Publication No. WO 00/07617.

Other specific additional therapeutic agents useful in the present methods for treating or preventing a Condition include, but are not limited to, rimonabant, 2-methyl-6-(phenylethynyl)-pyridine, 3[(2-methyl-1,4-thiazol-4-yl)ethynyl]pyridine, Melanotan-II, dexfenfluramine, fluoxetine, paroxetine, fenfluramine, fluvoxamine, sertaline, imipramine, desipramine, talsupram, nomifensine, leptin, nalmefene, 3-methoxynaltrexone, naloxone, nalterxone, butabindide, axokine, sibutramine, topiramate, phytopharm compound 57, Cerulenin, theophylline, pentoxifylline, zaprinast, sildenafil, aminone, milrinone, cilostamide, rolipram, cilomilast, phytanic acid, 4-[(E)-2-(5,6,7,8-tetramethyl-2-naphthalenyl)-1-propenyl]benzoic acid, retinoic acid, oleoyl-estrone, orlistat, lipstatin, tetrahydrolipstatin, teasaponin and diethylumbelliferyl phosphate.

In one embodiment, the additional therapeutic agent is an antiobesity agent.

Non-limiting examples of antiobesity agents useful in the present methods for treating or preventing a Condition include include an appetite suppressant, a metabolic rate enhancers and a nutrient absorption inhibitor.

Non-limiting examples of appetite suppressants useful in the present combination therapies include cannabinoid receptor 1 (CB₁) antagonists or inverse agonists (e.g., rimonabant); Neuropeptide Y (NPY1, NPY2, NPY4 and NPY5) antagonists; metabotropic glutamate subtype 5 receptor (mGluR5) antagonists (e.g., 2-methyl-6-(phenylethynyl)-pyridine and 3[(2-methyl-1,4-thiazol-4-yl)ethynyl]pyridine); melanin-concentrating hormone receptor (MCH1R and MCH2R) antagonists; melanocortin receptor agonists (e.g., Melanotan-II and Mc4r agonists); serotonin uptake inhibitors (e.g., dexfenfluramine and fluoxetine); serotonin (5HT) transport inhibitors (e.g., paroxetine, fluoxetine, fenfluramine, fluvoxamine, sertaline and imipramine); norepinephrine (NE) transporter inhibitors (e.g., desipramine, talsupram and nomifensine); ghrelin antagonists; leptin or derivatives thereof; opioid antagonists (e.g., nalmefene, 3-methoxynaltrexone, naloxone and nalterxone); orexin antagonists; bombesin receptor subtype 3 (BRS3) agonists; Cholecystokinin-A (CCK-A) agonists; ciliary neurotrophic factor (CNTF) or derivatives thereof (e.g., butabindide and axokine); monoamine reuptake inhibitors (e.g., sibutramine); glucagons-like peptide 1 (GLP-1) agonists; topiramate; and phytopharm compound 57.

Non-limiting examples of metabolic rate enhancers useful in the present combination therapies include acetyl-CoA carboxylase-2 (ACC2) inhibitors; beta adrenergic receptor 3 (β3) agonists; diacylglycerol acyltransferase inhibitors (DGAT1 and DGAT2); fatty acid synthase (FAS) inhibitors (e.g., Cerulenin); phosphodiesterase (PDE) inhibitors (e.g., theophylline, pentoxifylline, zaprinast, sildenafil, aminone, milrinone, cilostamide, rolipram and cilomilast); thyroid hormone β agonists; uncoupling protein activators (UCP-1, 2 or 3) (e.g., phytanic acid, 4-[(E)-2-(5,6,7,8-tetramethyl-2-naphthalenyl)-1-propenyl]benzoic acid and retinoic acid); acyl-estrogens (e.g., oleoyl-estrone); glucocorticoid antagonists; 11-beta hydroxyl steroid dehydrogenase type 1 (11β HSD-1) inhibitors; melanocortin-3 receptor (Mc3r) agonists; and stearoyl-CoA desaturase-1 (SCD-1) compounds.

Non-limiting examples of nutrient absorption inhibitors useful in the present combination therapies include lipase inhibitors (e.g., orlistat, lipstatin, tetrahydrolipstatin, teasaponin and diethylumbelliferyl phosphate); fatty acid transporter inhibitors; dicarboxylate transporter inhibitors; glucose transporter inhibitors; and phosphate transporter inhibitors.

Specific examples of antiobesity agents useful in the present combination therapies include include rimonabant, 2-methyl-6-(phenylethynyl)-pyridine, 3[(2-methyl-1,4-thiazol-4-yl)ethynyl]pyridine, Melanotan-II, dexfenfluramine, fluoxetine, paroxetine, fenfluramine, fluvoxamine, sertaline, imipramine, desipramine, talsupram, nomifensine, leptin, nalmefene, 3-methoxynaltrexone, naloxone, nalterxone, butabindide, axokine, sibutramine, topiramate, phytopharm compound 57, Cerulenin, theophylline, pentoxifylline, zaprinast, sildenafil, aminone, milrinone, cilostamide, rolipram, cilomilast, phytanic acid, 4-[(E)-2-(5,6,7,8-tetramethyl-2-naphthalenyl)-1-propenyl]benzoic acid, retinoic acid, oleoyl-estrone, orlistat, lipstatin, tetrahydrolipstatin, teasaponin and diethylumbelliferyl phosphate.

In one embodiment, useful antiobesity agents include rimonabant, dexfenfluramine, fenfluramine, phentermine, leptin, nalmefene, axokine, sibutramine, topiramate, phytopharm compound 57, oleoyl-estrone and orlistat. Non-limiting examples of anti-obesity agents useful in the present methods for treating diabetes include a 5-HT2C agonist, such as lorcaserin; a neuropeptide Y antagonist; an MCR4 agonist; an MCH receptor antagonist; a protein hormone, such as leptin or adiponectin; an AMP kinase activator; and a lipase inhibitor, such as orlistat. Appetite suppressants are not considered to be within the scope of the anti-obesity agents useful in the present methods.

In one embodiment, the present combination therapies for treating or preventing diabetes comprise administering a compound of formula (I), an antidiabetic agent and/or an antiobesity agent.

In another embodiment, the present combination therapies for treating or preventing diabetes comprise administering a compound of formula (I) and an antidiabetic agent.

In another embodiment, the present combination therapies for treating or preventing diabetes comprise administering a compound of formula (I) and an anti-obesity agent.

In one embodiment, the present combination therapies for treating or preventing obesity comprise administering a compound of formula (I), an antidiabetic agent and/or an antiobesity agent.

In another embodiment, the present combination therapies for treating or preventing obesity comprise administering a compound of formula (I) and an antidiabetic agent.

In another embodiment, the present combination therapies for treating or preventing obesity comprise administering a compound of formula (I) and an anti-obesity agent.

In one embodiment, the other therapeutic agent is an analgesic agent.

Non-limiting examples of analgesic agents useful in the present methods for treating pain include acetaminophen, an NSAID, an opiate or a tricyclic antidepressant.

In one embodiment, the other analgesic agent is acetaminophen or an NSAID.

Non-limiting examples of NSAIDS useful in the present methods for treating pain include a salicylate, such as aspirin, amoxiprin, benorilate or diflunisal; an arylalkanoic acid, such as diclofenac, etodolac, indometacin, ketorolac, nabumetone, sulindac or tolmetin; a 2-arylpropionic acid (a “profen”), such as ibuprofen, carprofen, fenoprofen, flurbiprofen, loxoprofen, naproxen, tiaprofenic acid or suprofen; a fenamic acid, such as mefenamic acid or meclofenamic acid; a pyrazolidine derivative, such as phenylbutazone, azapropazone, metamizole or oxyphenbutazone; a coxib, such as celecoxib, etoricoxib, lumiracoxib or parecoxib; an oxicam, such as piroxicam, lornoxicam, meloxicam or tenoxicam; or a sulfonanilide, such as nimesulide.

In another embodiment, the other analgesic agent is an opiate.

Non-limiting examples of opiates useful in the present methods for treating pain include an anilidopiperidine, a phenylpiperidine, a diphenylpropylamine derivative, a benzomorphane derivative, an oripavine derivative and a morphinane derivative. Additional illustrative examples of opiates include morphine, diamorphine, heroin, buprenorphine, dipipanone, pethidine, dextromoramide, alfentanil, fentanyl, remifentanil, methadone, codeine, dihydrocodeine, tramadol, pentazocine, vicodin, oxycodone, hydrocodone, percocet, percodan, norco, dilaudid, darvocet or lorcet.

In another embodiment, the other analgesic agent is a tricyclic antidepressant.

Non-limiting examples of tricyclic antidepressants useful in the present methods for treating pain include amitryptyline, carbamazepine, gabapentin or pregabalin.

In another embodiment, the other therapeutic agent is an antihypertensive agent.

Non-limiting examples of antihypertensive agents useful in the present methods for treating a Condition include β-blockers and calcium channel blockers (for example diltiazem, verapamil, nifedipine, amlopidine, and mybefradil), ACE inhibitors (for example captopril, lisinopril, enalapril, spirapril, ceranopril, zefenopril, fosinopril, cilazopril, and quinapril), AT-1 receptor antagonists (for example losartan, irbesartan, and valsartan), renin inhibitors and endothelin receptor antagonists (for example sitaxsentan).

The Oxypiperidine Derivatives can be combined with an H₁ receptor antagonist (i.e., the Oxypiperidine Derivatives can be combined with an H₁ receptor antagonist in a pharmaceutical composition, or the Oxypiperidine Derivatives can be administered with one or more H₁ receptor antagonists).

Numerous chemical substances are known to have histamine H₁ receptor antagonist activity and can therefore be used in the methods of this invention. Many H₁ receptor antagonists useful in the methods of this invention can be classified as ethanolamines, ethylenediamines, alkylamines, phenothiazines or piperidines. Representative H₁ receptor antagonists include, without limitation: astemizole, azatadine, azelastine, acrivastine, brompheniramine, cetirizine, chlorpheniramine, clemastine, cyclizine, carebastine, cyproheptadine, carbinoxamine, descarboethoxyloratadine, diphenhydramine, doxylamine, dimethindene, ebastine, epinastine, efletirizine, fexofenadine, hydroxyzine, ketotifen, loratadine, levocabastine, meclizine, mizolastine, mequitazine, mianserin, noberastine, norastemizole, picumast, pyrilamine, promethazine, terfenadine, tripelennamine, temelastine, trimeprazine and triprolidine. Other compounds can readily be evaluated to determine activity at H₁ receptors by known methods, including specific blockade of the contractile response to histamine of isolated guinea pig ileum. See for example, WO98/06394 published Feb. 19, 1998.

Those skilled in the art will appreciate that the H₁ receptor antagonist is used at its known therapeutically effective dose, or the H₁ receptor antagonist is used at its normally prescribed dosage.

In one embodiment, the H₁ receptor antagonist is selected from: astemizole, azatadine, azelastine, acrivastine, brompheniramine, cetirizine, chlorpheniramine, clemastine, cyclizine, carebastine, cyproheptadine, carbinoxamine, descarboethoxyloratadine, diphenhydramine, doxylamine, dimethindene, ebastine, epinastine, efletirizine, fexofenadine, hydroxyzine, ketotifen, loratadine, levocabastine, meclizine, mizolastine, mequitazine, mianserin, noberastine, norastemizole, picumast, pyrilamine, promethazine, terfenadine, tripelennamine, temelastine, trimeprazine or triprolidine.

In another embodiment, the H₁ receptor antagonist is selected from: astemizole, azatadine, azelastine, brompheniramine, cetirizine, chlorpheniramine, clemastine, carebastine, descarboethoxyloratadine, diphenhydramine, doxylamine, ebastine, fexofenadine, loratadine, levocabastine, mizolastine, norastemizole, or terfenadine.

In another embodiment, the H₁ receptor antagonist is selected from: azatadine, brompheniramine, cetirizine, chlorpheniramine, carebastine, descarboethoxy-loratadine, diphenhydramine, ebastine, fexofenadine, loratadine, or norastemizole.

In still another embodiment, the H₁ antagonist is selected from loratadine, descarboethoxyloratadine, fexofenadine or cetirizine. In a further embodiment, the H₁ antagonist is loratadine or descarboethoxyloratadine.

In one embodiment, the H₁ receptor antagonist is loratadine.

In another embodiment, the H₁ receptor antagonist is descarboethoxyloratadine.

In still another embodiment, the H₁ receptor antagonist is fexofenadine.

In yet another embodiment, the H₁ receptor antagonist is cetirizine.

In one embodiment, the present methods of treating an allergy-induced airway response in a patient further comprise administering to the patient an H₁ receptor antagonist.

In another embodiment, the present methods of treating allergy in a patient further comprise administering to the patient an H₁ receptor antagonist.

In another embodiment, the present methods of treating congestion in a patient further comprise administering to the patient an H₁ receptor antagonist. In one embodiment, the congestion is nasal congestion.

In the methods of this invention wherein a combination of an Oxypiperidine Derivative of this invention (compound of formula I) is administered with a H₁ antagonist, the antagonists can be administered simultaneously or sequentially (first one and then the other over a period of time). In general, when the antagonists are administered sequentially, the H₃ antagonist of this invention (compound of formula I) is administered first.

When administering a combination therapy to a patient in need of such administration, the therapeutic agents in the combination, or a composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts).

In one embodiment, the one or more Oxypiperidine Derivatives are administered during a time when the additional therapeutic agent(s) exert their prophylactic or therapeutic effect, or vice versa.

In another embodiment, the one or more Oxypiperidine Derivatives and the additional therapeutic agent(s) are administered in doses commonly employed when such agents are used as monotherapy for treating a Condition.

In another embodiment, the one or more Oxypiperidine Derivatives and the additional therapeutic agent(s) are administered in doses lower than the doses commonly employed when such agents are used as monotherapy for treating a Condition.

In still another embodiment, the one or more Oxypiperidine Derivatives and the additional therapeutic agent(s) act synergistically and are administered in doses lower than the doses commonly employed when such agents are used as monotherapy for treating a Condition.

In one embodiment, the one or more Oxypiperidine Derivatives and the additional therapeutic agent(s) are present in the same composition. In one embodiment, this composition is suitable for oral administration. In another embodiment, this composition is suitable for intravenous administration.

The one or more Oxypiperidine Derivatives and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

In one embodiment, the administration of one or more Oxypiperidine Derivatives and the additional therapeutic agent(s) may inhibit the resistance of a Condition to one or more of these agents.

In one embodiment, the additional therapeutic agent is used at its known therapeutically effective dose. In another embodiment, the additional therapeutic agent is used at its normally prescribed dosage. In another embodiment, the additional therapeutic agent is used at less than its normally prescribed dosage or its known therapeutically effective dose.

The doses and dosage regimen of the other agents used in the combination therapies of the present invention for the treatment or prevention of a Condition can be determined by the attending clinician, taking into consideration the approved doses and dosage regimen in the package insert; the age, sex and general health of the patient; and the type and severity of the viral infection or related disease or disorder. When administered in combination, the Oxypiperidine Derivative(s) and the other agent(s) for treating diseases or conditions listed above can be administered simultaneously or sequentially. This particularly useful when the components of the combination are given on different dosing schedules, e.g., one component is administered once daily and another every six hours, or when the compositions are different, e.g. one is a tablet and one is a capsule. A kit comprising the separate dosage forms is therefore advantageous.

Generally, a total daily dosage of the one or more Oxypiperidine Derivatives and the additional therapeutic agent(s) can when administered as combination therapy, range from about 0.1 to about 2000 mg per day, although variations will necessarily occur depending on the target of the therapy, the patient and the route of administration. In one embodiment, the dosage is from about 0.2 to about 100 mg/day, administered in a single dose or in 2-4 divided doses. In another embodiment, the dosage is from about 1 to about 500 mg/day, administered in a single dose or in 2-4 divided doses. In another embodiment, the dosage is from about 1 to about 200 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In a further embodiment, the dosage is from about 1 to about 20 mg/day, administered in a single dose or in 2-4 divided doses.

### Compositions and Administration

In one embodiment, the invention provides compositions comprising an effective amount of one or more Oxypiperidine Derivatives or a pharmaceutically acceptable salt, solvate, ester or prodrug thereof, and a pharmaceutically acceptable carrier.

For preparing compositions comprising one or more Oxypiperidine Derivatives, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

In one embodiment, an Oxypiperidine Derivative is administered orally.

In one embodiment, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation is from about 0.1 to about 2000 mg. Variations will necessarily occur depending on the target of the therapy, the patient and the route of administration. In one embodiment, the unit dose dosage is from about 0.2 to about 1000 mg. In another embodiment, the unit dose dosage is from about 1 to about 500 mg. In another embodiment, the unit dose dosage is from about 1 to about 100 mg/day. In still another embodiment, the unit dose dosage is from about 1 to about 50 mg. In yet another embodiment, the unit dose dosage is from about 1 to about 10 mg.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, the condition and size of the patient, as well as severity of the symptoms being treated. A typical recommended daily dosage regimen for oral administration can range from about 1 mg/day to about 1000 mg/day, 1 mg/day to about 500 mg/day, 1 mg/day to about 300 mg/day, 1 mg/day to about 75 mg/day, 1 mg/day to about 50 mg/day, or 1 mg/day to about 20 mg/day, in one dose or in two to four divided doses.

When the invention comprises a combination of one or more Oxypiperidine Derivatives and an additional therapeutic agent, the two active components may be co-administered simultaneously or sequentially, or a single composition comprising one or more Oxypiperidine Derivatives and the additional therapeutic agent(s) in a pharmaceutically acceptable carrier can be administered. The components of the combination can be administered individually or together in any conventional dosage form such as capsule, tablet, powder, cachet, suspension, solution, suppository, nasal spray, etc. The dosage of the additional therapeutic agent can be determined from published material, and may range from about 1 to about 1000 mg per dose. In one embodiment, when used in combination, the dosage levels of the individual components are lower than the recommended individual dosages because of an advantageous effect of the combination.

In one embodiment, the components of a combination therapy regimen are to be administered simultaneously, they can be administered in a single composition with a pharmaceutically acceptable carrier.

In another embodiment, when the components of a combination therapy regimen are to be administered separately or sequentially, they can be administered in separate compositions, each containing a pharmaceutically acceptable carrier.

### Kits

In one aspect, the present invention provides a kit comprising an effective amount of one or more Oxypiperidine Derivatives, or a pharmaceutically acceptable salt, solvate, ester or prodrug thereof, and a pharmaceutically acceptable carrier.

In another aspect the present invention provides a kit comprising an amount of one or more Oxypiperidine Derivatives, or a pharmaceutically acceptable salt, solvate, ester or prodrug thereof, and an amount of at least one additional therapeutic agent listed above, wherein the combined amounts are effective for treating or preventing a Condition in a patient.

When the components of a combination therapy regimen are to be administered in more than one composition, they can be provided in a kit comprising a single package containing one or more containers, wherein one container contains one or more Oxypiperidine Derivatives in a pharmaceutically acceptable carrier, and a second, separate container comprises an additional therapeutic agent in a pharmaceutically acceptable carrier, with the active components of each composition being present in amounts such that the combination is therapeutically effective.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

1. A compound having the structure: 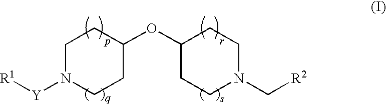
or a pharmaceutically acceptable salt, solvate, ester or prodrug thereof, wherein:
Y is a bond, -alkylene-, —C(O)—, —OC(O)— or —NHC(O)—;
R¹ is aryl, cycloalkyl, cycloalkenyl, heterocycloalkyl, heterocycloalkenyl or heteroaryl, wherein an aryl, cycloalkyl, cycloalkenyl, heterocycloalkyl, heterocycloalkenyl or heteroaryl group can be optionally substituted with up to 3 substituents, which can be the same or different, and are selected from alkyl, —O-alkyl, halo, haloalkyl, —O-haloalkyl, —CN, —C(O)OR³, —N(R⁴)₂, —C(O)N(R⁴)₂, —C(O)R⁵, —NHC(O)R⁵, —NHS(O)₂R³ or —S(O)₂N(R⁴)₂, and wherein R¹ is cycloalkyl, the cycloalkyl group can be optionally fused to an aryl or heteroaryl ring;
R² is aryl, heterocycloalkyl, heterocycloalkenyl or heteroaryl, wherein an aryl, heterocycloalkyl, heterocycloalkenyl or heteroaryl group can be optionally substituted with up to 3 substituents, which can be the same or different, and are selected from alkyl, cycloalkyl, heterocycloalkyl, heteroaryl, —O-alkyl, —O-aryl, halo, haloalkyl, —O-haloalkyl, —CN, —OC(O)R⁵, —C(O)OR³, —N(R⁴)₂, —C(O)N(R⁴)₂, —C(O)R⁵, —NHC(O)R⁵, —NHS(O)₂R³ or —S(O)₂N(R⁴)₂;
each occurrence of R³ is independently H, alkyl, aryl, cycloalkyl, heterocycloalkyl or heteroaryl;
each occurrence of R⁴ is independently H, alkyl, aryl, cycloalkyl, heterocycloalkyl or heteroaryl;
each occurrence of R⁵ is independently H, alkyl, aryl, cycloalkyl, cycloalkenyl, heterocycloalkyl, heterocycloalkenyl, heteroaryl, —O-alkyl, —NH-alkyl, —O-aryl or —NH-aryl;
p is an integer ranging from 0 to 2;
q is an integer ranging from 0 to 2;
r is an integer ranging from 0 to 2; and
s is an integer ranging from 0 to 2.

2. The compound of claim 1 wherein r and s are each 1.

3. The compound of claim 2, wherein p and q are each 1.

4. The compound of claim 2, wherein the sum of p and q is 1.

5. (canceled)

6. The compound of claim 1, wherein Y is a bond, —CH₂, or —C(O)—.

7. The compound of claim 1, wherein R¹ is cycloalkyl, aryl or heteroaryl.

8. (canceled)

9. The compound of claim 1, wherein R² is heteroaryl or heterocycloalkyl.

10. (canceled)

11. The compound of claim 7, wherein R² is heteroaryl or heterocycloalkyl.

12. The compound of claim 11, wherein R¹ is: 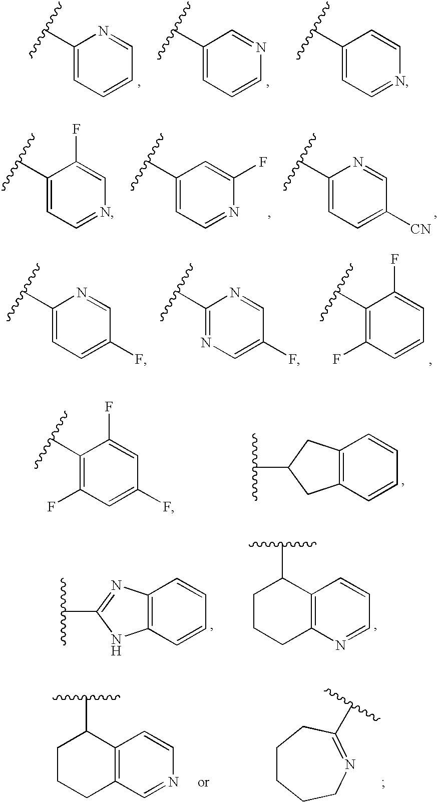
and R² is:

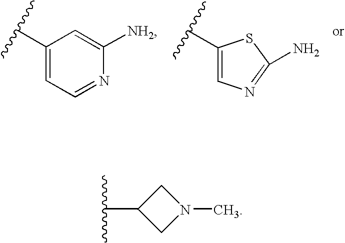

13. (canceled)

14. The compound of claim 6, wherein R¹ is cycloalkyl, aryl or heteroaryl and R² is heteroaryl or heterocycloalkyl.

15. (canceled)

16. The compound of claim 1, having the formula: 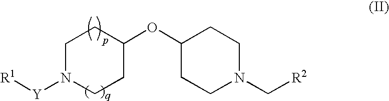
or a pharmaceutically acceptable salt, solvate, ester or prodrug thereof, wherein:
Y is a bond, -alkylene-, —C(O)— or —NHC(O)—;
R¹ is aryl, cycloalkyl, heterocycloalkyl or heteroaryl, wherein an aryl, cycloalkyl, heterocycloalkyl or heteroaryl group can be optionally substituted with up to 3 substituents, which can be the same or different, and are selected from alkyl, halo, haloalkyl, —CN and —N(R⁴)₂;
R² is heterocycloalkyl or heteroaryl, either of which can be optionally substituted with up to 3 substituents, which can be the same or different, and are selected from alkyl, halo, haloalkyl, —CN and —N(R⁴)₂;
each occurrence of R⁴ is independently H or alkyl;
p is an integer ranging from 0 to 2; and
q is an integer ranging from 0 to 2.

17. The compound of claim 16, wherein Y is a bond, —CH₂— or —C(O)—.

18. (canceled)

19. The compound of claim 17, wherein R¹ is:

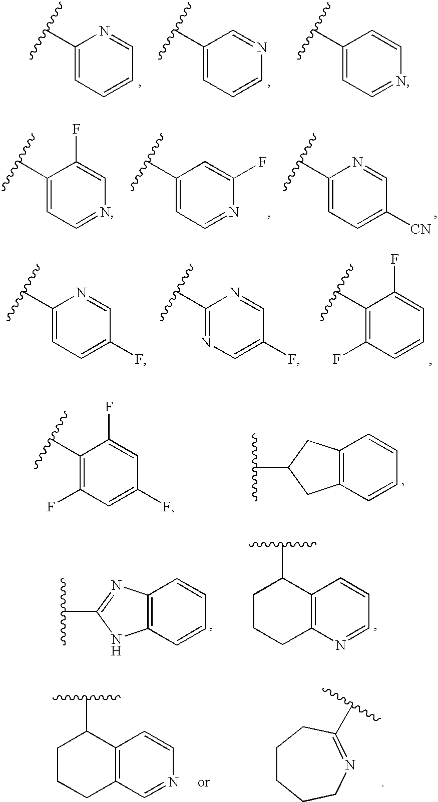

20. The compound of claim 19, wherein R² is:

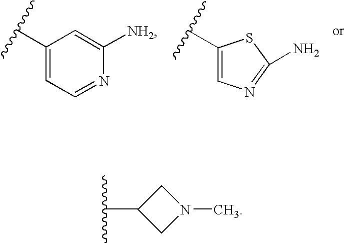

21. The compound of claim 16, wherein p and q are each 1.

22. The compound of claim 20, wherein p and q are each 1.

23. A compound having the structure: 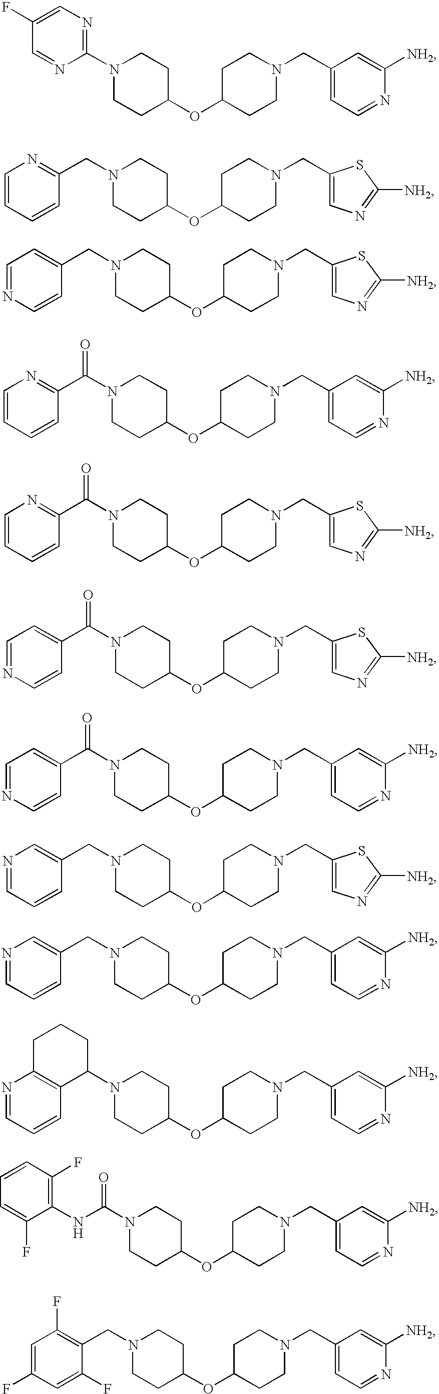 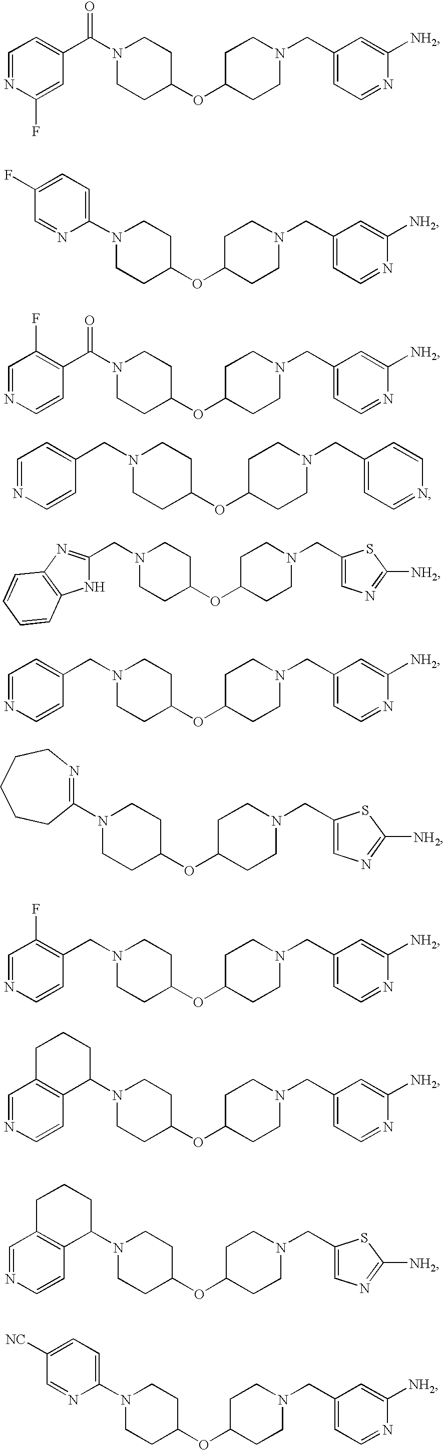 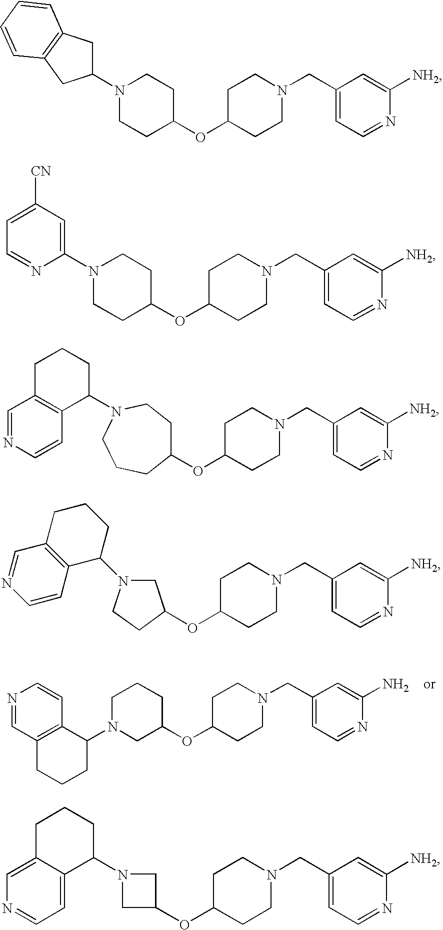
or a pharmaceutically acceptable salt, solvate, ester or prodrug thereof.

24. A composition comprising an effective amount of one or more compounds of claim 1 and a pharmaceutically acceptable carrier.

25. (canceled)

26. (canceled)

27. (canceled)

28. A method of treating allergy, an allergy-induced airway response, congestion, hypotension, a cardiovascular disease, a gastrointestinal disorder, obesity, a sleep disorder, pain, diabetes, a diabetic complication, impaired glucose tolerance, impaired fasting glucose or a central nervous system disorder in a patient, comprising administering to the patient an effective amount of a compound of claim 1 or a pharmaceutically acceptable salt, solvate, ester or prodrug thereof.

29. The method of claim 28, further comprising administering to the patient at least one additional therapeutic agent, which is not a compound of claim 1, and wherein the at least one additional therapeutic agent(s) are selected from an antiobesity agent, an antidiabetic agent, an agent useful for treating a cardiovascular disease, an agent useful for treating a gastrointestinal disorder, an agent useful for treating allergy or an allergy-induced airway response, an agent useful for treating congestion, an agent useful for treating a CNS disorder, an agent useful for treating hypotension, an analgesic agent or an agent useful for treating a sleep disorder.

30. (canceled)

31. (canceled)

32. (canceled)

33. (canceled)
